(19) United States
(12) Patent Application Publication
(10) Pub. No.: US 2022/0241312 A1
(43) Pub. Date: Aug. 4, 2022

(54) COMPOUNDS FOR TREATING CANCER

(71) Applicant: Tems Pharmaceuticals, Inc., Foster City, CA (US)

(72) Inventors: Martijn FENAUX, Foster City, CA (US); Weidong ZHONG, Foster City, CA (US); Thorsten A. KIRSCHBERG, Foster City, CA (US)

(21) Appl. No.: 17/594,943

(22) PCT Filed: May 1, 2020
(86) PCT No.: PCT/US2020/031132

§ 371 Date: Nov. 3, 2021

Related U.S. Application Data
(60) Provisional application No. 62/843,005, filed on May 3, 2019.

(51) Int. Cl. A61K 31/7072; C07H 19/10; A61K 39/395; A61K 31/47; A61K 31/44; A61K 31/519; A61P 35/00

(52) U.S. Cl. CPC A61K 31/7072; C07H 19/10; A61K 39/3955; A61P 35/00; A61K 31/44; A61K 31/519; A61K 31/47

(57) ABSTRACT

Provided herein are prodrugs of 5-fluorodeoxyuridine monophosphate compounds, compositions thereof, methods of their preparation, and their use in treating cancers. In another aspect, provided herein is a pharmaceutical composition comprising any compound disclosed herein, or a pharmaceutically acceptable salt thereof, and at least one pharmaceutically acceptable carrier, diluent, and excipient.

### CROSS-REFERENCE TO RELATED APPLICATION

This application claims priority to U.S. Provisional Application No. 62/843,005, filed on May 3, 2019, the content of which is incorporated herein by reference in its entirety.

### FIELD

The present disclosure relates generally to prodrugs of 5-fluorodeoxyuridine monophosphate compounds, compositions thereof, methods of their preparation, and their use in treating cancers.

### BACKGROUND

5-Fluorouracil (5-FU) is a chemotherapeutic agent extensively used in the treatment of various cancers. Classified as an antimetabolite, 5-FU is intracellularly metabolized to 5-fluorouridine-5′-triphosphate (5-FUTP) and 5-fluoro-2′-deoxyuridine-5′-triphosphate (5-FdUTP), pharmacodynamically active metabolites that can be incorporated into RNA and DNA, respectively, resulting in cell death (see FIG. 1). Additionally, 5-FU is metabolized to 5-fluoro-2′-deoxyuridine-5′-monophosphate (5-FdUMP), which is believed to inhibit the activity of thymidylate synthase (TS), interrupting the production of DNA, and eventually inducing cell death (Malet-Martino et al., (2002) Oncologist, 7:288-323; see FIG. 1).

However, 80-85% of 5-FU is catabolized by the enzyme dihydropyrimidine dehydrogenase (DPD) to pharmacodynamically inactive metabolites, which contributes to the neurotoxicity and cardiotoxicity of a 5-FU-based treatment regimen. This catabolization contributes to the low half-life (5-20 min) of 5-FU and limited availability in vivo, which constitutes a major drawback of its use as a chemotherapeutic agent. Additionally, the activity level of DPD in patients can vary widely, thus making the bioavailability of 5-FU unpredictable, and can result in severe and even fatal 5-FU toxicity. 5-FU is typically administered by either bolus injections or continuous infusion. However, bolus injection is associated with myelotoxicity, whereas continuous infusion is associated with palmar-plantar erythrodysesthesia, stomatitis, neurotoxicity, and cardiotoxicity. Furthermore, both intravenous modes of administration have elevated risk of infection at the injection site, nausea, diarrhea, alopecia, and dermatitis (Malet-Martino et al., (2002) Oncologist, 7:288-323). Oral administration of 5-FU has been investigated, but it is plagued by unpredictable absorption due the varying levels of DPD in the gastrointestinal tract (Cao et al., (1994) Cancer Res, 54:1507-1510).

Prodrugs of 5-FU, such as capecitabine, ftorafur plus uracil (UFT), and ftorafur plus 5-chloro-2,4-dihydroxypyridine plus potassium oxonate (S-1), have been developed to permit oral administration and to attempt to address the many drawbacks associated with 5-FU discussed above. However, although S-1 and capecitabine can be administered orally, switching 5-FU for either S-1 or capecitabine does not result in any significant changes in either efficacy or adverse events. Another disadvantage of capecitabine and S-1 is the required twice-daily oral administration. Therefore, the efficacy of these drugs is highly dependent on patient compliance (Malet-Martino et al., (2002) Oncologist, 7:288-323). In addition, ftorafur-based treatment regimens can lead to certain gastrointestinal-related side effects. Accordingly, there is a need for improved chemotherapeutic derivatives of 5-FU to treat a variety of cancers.

The compounds disclosed herein are prodrugs of 5-fluorodeoxyuridine monophosphate (FUdR-MP or 5-FdUMP). In contrast to the significant catabolization observed for 5-FU, many catabolic pathways are not accessible for the compounds disclosed herein. Release of the active monophosphate from the prodrugs disclosed herein is a simple two-step process involving deprotection and ring-opening of the cyclized phosphate.

### BRIEF SUMMARY

In one aspect, provided is a compound of formula (I)

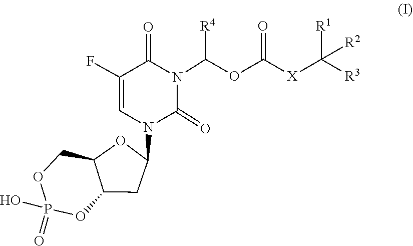

or a pharmaceutically acceptable salt thereof, wherein:
R¹ is H, optionally substituted C₁-C₆ alkyl, optionally substituted C₃-C₆ cycloalkyl, optionally substituted C₆-C₁₀ aryl, or optionally substituted 5- to 10-membered heteroaryl;
or R¹ and one R⁵ group are taken together with the atoms to which they are attached to form an optionally substituted 5- to 7-membered heterocyclyl;
R² is H, optionally substituted C₁-C₆ alkyl, —OR⁵, or —N(R⁵)₂;
or R¹ and R² are taken together with the carbon atom to which they are attached to form an optionally substituted 4- to 7-membered heterocyclyl or an optionally substituted C₃-C₆ cycloalkyl;
R³ is H or optionally substituted C₁-C₆ alkyl;
R⁴ is H, optionally substituted C₁-C₆ alkyl, or optionally substituted C₃-C₆ cycloalkyl;
X is O, C(R⁶)₂, or a bond;
each R⁵ is independently H, optionally substituted C₁-C₆ alkyl, or optionally substituted C₃-C₆ cycloalkyl;
or two R⁵ groups are taken together with the nitrogen atom to which they are attached to form an optionally substituted 5- to 7-membered heterocyclyl; and
each R⁶ is independently H, optionally substituted C₁-C₆ alkyl, or optionally substituted C₃-C₆ cycloalkyl;
or two R⁶ groups are taken together with the carbon atom to which they are attached to form an optionally substituted C₃-C₆ cycloalkyl or an optionally substituted 3- to 7-membered heterocyclyl.

In some embodiments, X is O. In some embodiments, X is a bond. In some embodiments, X is C(R⁶)₂.

In some embodiments, each R⁶ is independently H; C₁-C₆ alkyl optionally substituted with halogen, —OH, —CN, or —NH₂; or C₃-C₆ cycloalkyl optionally substituted with halogen, —OH, —CN, —NH₂, C₁-C₆ alkyl, or C₁-C₆ haloalkyl; or two R⁶ groups are taken together with the carbon atom to which they are attached to form a C₃-C₆ cycloalkyl or a 3- to 7-membered heterocyclyl, each of which is optionally substituted with halogen, —OH, —CN, —NH₂, C₁-C₆ alkyl, or C₁-C₆ haloalkyl. In some embodiments, each R⁶ is independently H or unsubstituted C₁-C₃ alkyl. In some embodiments, each R⁶ is independently H or —CH₃.

In some embodiments, R¹ is H; C₁-C₆ alkyl optionally substituted with halogen, —OH, —CN, or —NH₂; or C₃-C₆ cycloalkyl, C₆-C₁₀ aryl, or 5- to 10-membered heteroaryl, each of which is optionally substituted with halogen, —OH, —CN, —NH₂, C₁-C₆ alkyl, or C₁-C₆ haloalkyl, or R¹ and one R⁵ group are taken together with the atoms to which they are attached to form a 5- to 7-membered heterocyclyl optionally substituted with halogen, —OH, —NH₂, C₁-C₆ alkyl, or C₁-C₆ haloalkyl. In some embodiments, R¹ is H or unsubstituted C₁-C₃ alkyl, or R¹ and one R⁵ group are taken together with the atoms to which they are attached to form an unsubstituted 5- to 6-membered heterocyclyl. In some embodiments, R¹ is H or —CH₃, or R¹ and one R⁵ group are taken together with the atoms to which they are attached to form an unsubstituted 5-membered heterocyclyl.

In some embodiments, each R⁵ is independently H; C₁-C₆ alkyl optionally substituted with halogen, —OH, —CN, or —NH₂; or C₃-C₆ cycloalkyl optionally substituted with halogen, —OH, —CN, —NH₂, C₁-C₆ alkyl, or C₁-C₆ haloalkyl; or two R⁵ groups are taken together with the nitrogen atom to which they are attached to form a 5- to 7-membered heterocyclyl optionally substituted with halogen, —OH, —CN, —NH₂, C₁-C₆ alkyl, or C₁-C₆ haloalkyl, wherein the 5- to 7-membered heterocyclyl optionally contains 1-2 additional ring heteroatoms selected from the group consisting of O, N, S, S(═O), and S(═O)₂.

In some embodiments, R² is H; C₁-C₆ alkyl optionally substituted with halogen, —OH, —CN, or —NH₂; —OR⁵; or —N(R⁵)₂. In some embodiments, R² is H, unsubstituted C₁-C₃ alkyl, —O(unsubstituted C₁-C₆ alkyl), —OH, —NH₂, or —NH(unsubstituted C₁-C₆ alkyl). In some embodiments, R² is H, —CH₃, —OCH₃, —OH, —NH₂, or —N(H)CH₃.

In some embodiments, R¹ and R² are taken together with the carbon atom to which they are attached to form a 4- to 7-membered heterocyclyl or C₃-C₆ cycloalkyl, each of which is optionally substituted with halogen, —OH, —CN, —NH₂, C₁-C₆ alkyl, or C₁-C₆ haloalkyl. In some embodiments, R¹ and R² are taken together with the carbon atom to which they are attached to form an unsubstituted 5- to 6-membered heterocyclyl. In some embodiments, the 5- to 6-membered heterocyclyl is unsubstituted pyrrolidinyl.

In some embodiments, R³ is H; or C₁-C₆ alkyl optionally substituted with halogen, —OH, —CN, or —NH₂. In some embodiments, R³ is H or unsubstituted C₁-C₃ alkyl. In some embodiments, R³ is H or —CH₃.

In some embodiments, R⁴ is H; C₁-C₆ alkyl optionally substituted with halogen, —OH, —CN, or —NH₂; or C₃-C₆ cycloalkyl optionally substituted with halogen, —OH, —CN, —NH₂, C₁-C₆ alkyl, or C₁-C₆ haloalkyl. In some embodiments, R⁴ is H or unsubstituted C₁-C₃ alkyl. In some embodiments, R⁴ is H or —CH₃.

Also provided herein is a compound which is selected from the group consisting of:

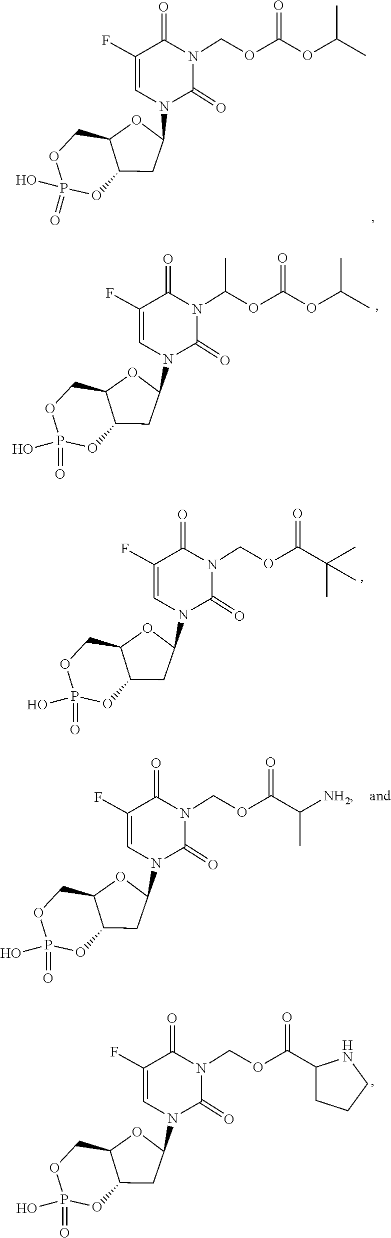

or a pharmaceutically acceptable salt thereof.

In another aspect, provided herein is a pharmaceutical composition comprising any compound disclosed herein, or a pharmaceutically acceptable salt thereof, and at least one pharmaceutically acceptable carrier, diluent, and excipient.

In a further aspect, provided herein is a method of treating cancer in a patient in need thereof, comprising administering to the patient a therapeutically effective amount of any compound disclosed herein, or a pharmaceutically acceptable salt thereof, or a therapeutically effective amount of the pharmaceutical composition disclosed herein. In some embodiments, the cancer is liver, colorectal, anal, breast, gastrointestinal, skin, stomach, esophageal, or pancreatic cancer. In some embodiments, the cancer originates from the liver or spreads to the liver. In some embodiments, the method further comprises administering one or more additional pharmaceutical agents. In some embodiments, the one or more additional pharmaceutical agents is selected from the group consisting of cabozantinib-S-malate, pembrolizumab, lenvatinib mesylate, sorafenib tosylate, nivolumab, and regorafenib. In some embodiments, the one or more additional pharmaceutical agents is leucovorin.

### BRIEF DESCRIPTION OF THE DRAWINGS

FIG. 1 shows the metabolism of 5-FU.

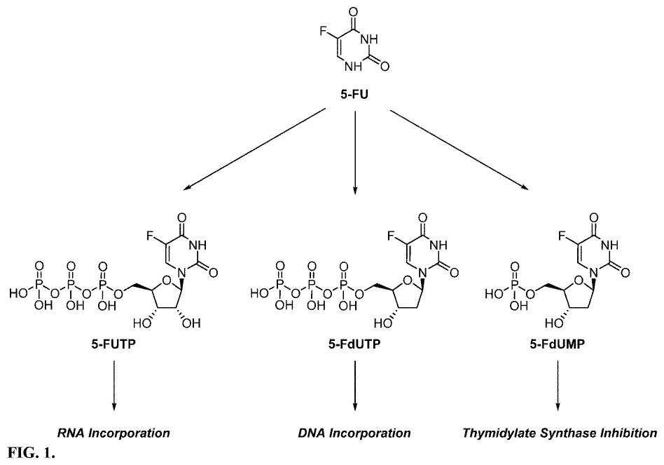

### DETAILED DESCRIPTION

### Definitions

As used herein, the following definitions shall apply unless otherwise indicated. Further, if any term or symbol used herein is not defined as set forth below, it shall have its ordinary meaning in the art.

“Comprising” is intended to mean that the compositions and methods include the recited elements, but not excluding others. “Consisting essentially of” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amount of, e.g., other ingredients and substantial method steps recited. Embodiments defined by each of these transition terms are within the scope of this invention. Thus, it is understood that aspects and embodiments described herein as “comprising” include “consisting of” and “consisting essentially of” embodiments.

“Effective amount” or dose of a compound or a composition, refers to that amount of the compound, or a pharmaceutically acceptable salt thereof, or the composition that results in an intended result as desired based on the disclosure herein. Effective amounts can be determined by standard pharmaceutical procedures in cell cultures or experimental animals, e.g., and without limitation, by determining the LD₅₀ (the dose lethal to 50% of the population) and the ED₅₀ (the dose therapeutically effective in 50% of the population). In some variations, the desired result or outcome is due to one or more metabolites of an administered compound.

The term “excipient” as used herein means an inert or inactive substance that may be used in the production of a drug or pharmaceutical, such as a tablet containing a compound of the invention as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

“Patient” refers to mammals and includes humans and non-human mammals. Examples of patients include, but are not limited to mice, rats, hamsters, guinea pigs, pigs, rabbits, cats, dogs, goats, sheep, cows, and humans. In some embodiments, patient refers to a human.

“Pharmaceutically acceptable” refers to safe and non-toxic, preferably for in vivo, more preferably, for human administration.

“Pharmaceutically acceptable salt” refers to a salt that is pharmaceutically acceptable. A compound described herein may be administered as a pharmaceutically acceptable salt.

“Prodrug” refers to a compound that, after administration, is metabolized or otherwise converted to a biologically active or more active compound (or drug) with respect to at least one property. A prodrug, relative to the drug, is modified chemically in a manner that renders it, relative to the drug, less active or inactive, but the chemical modification is such that the corresponding drug is generated by metabolic or other biological processes after the prodrug is administered. A prodrug may have, relative to the active drug, altered metabolic stability or transport characteristics, fewer side effects or lower toxicity, or improved flavor (for example, see the reference Nogrady, 1985, Medicinal Chemistry A Biochemical Approach, Oxford University Press, New York, pages 388-392, incorporated herein by reference). A prodrug may be synthesized using reactants other than employing the corresponding drug. For illustration and without limitation, prodrugs include, carboxy esters, linear and cyclic phosphate esters and phosphoramide and phosphoramidates, carbamates, preferably phenolic carbamates (i.e., carbamates where the hydroxy group is part of an aryl or heteroaryl moiety, where the aryl and heteroaryl may be optionally substituted), and the like.

“Salt” refers to an ionic compound formed between an acid and a base. When the compound provided herein contains an acidic functionality, such salts include, without limitation, alkali metal, alkaline earth metal, and ammonium salts. As used herein, ammonium salts include, salts containing protonated nitrogen bases and alkylated nitrogen bases. Exemplary and non-limiting cations useful in pharmaceutically acceptable salts include Na, K, Rb, Cs, NH₄, Ca, Ba, imidazolium, and ammonium cations based on naturally occurring amino acids. When the compounds utilized herein contain basic functionality, such salts include, without limitation, salts of organic acids, such as carboxylic acids and sulfonic acids, and mineral acids, such as hydrogen halides, sulfuric acid, phosphoric acid, and the like. Exemplary and non-limiting anions useful in pharmaceutically acceptable salts include oxalate, maleate, acetate, propionate, succinate, tartrate, chloride, sulfate, bisulfate, mono-, di-, and tribasic phosphate, mesylate, tosylate, and the like.

“Therapeutically effective amount” or dose of a compound or a composition refers to that amount of the compound or the composition that results in reduction or inhibition of symptoms or a prolongation of survival in a patient. The results may require multiple doses of the compound or the composition. In some variations, the desired therapeutic outcome is due to one or more metabolites of an administered compound.

As used herein, “treatment” or “treating” is an approach for obtaining beneficial or desired results including clinical results. For purposes of this invention, beneficial or desired results include, but are not limited to, one or more of the following: decreasing one more symptoms resulting from the disease or disorder, diminishing the extent of the disease or disorder, stabilizing the disease or disorder (e.g., preventing or delaying the worsening of the disease or disorder), delaying the occurrence or recurrence of the disease or disorder, delay or slowing the progression of the disease or disorder, ameliorating the disease or disorder state, providing a remission (whether partial or total) of the disease or disorder, decreasing the dose of one or more other medications required to treat the disease or disorder, enhancing the effect of another medication used to treat the disease or disorder, delaying the progression of the disease or disorder, increasing the quality of life, and/or prolonging survival of a patient. Also encompassed by “treatment” is a reduction of pathological consequence of the disease or disorder. The methods of the invention contemplate any one or more of these aspects of treatment.

An “isotopomer” of a compound is a compound in which one or more atoms of the compound have been replaced with isotopes of those same atoms. For example, where H has been replaced by D or T, or ¹²C has been replaced by ¹¹C or ¹⁴N has been replaced by ¹⁵N. For example, and without limitation, replacement of with D can in some instances lead to reduced rates of metabolism and therefore longer half-lives. Replacement of H with T can provide radioligands potentially useful in binding studies. Replacement of ¹²C with the short-lived isotope ¹¹C can provide ligands useful in Positron Emission Tomography (PET) scanning. Replacement of ¹⁴N with ¹⁵N provides compounds that can be detected/monitored by ¹⁵N NMR spectroscopy. For example, an isotopomer of a compound containing —CH₂CH₃ is that compound but containing —CD₂CD₃ instead of the —CH₂CH₃.

“Stereoisomer” or “stereoisomers” refer to compounds that differ in the stereogenicity of the constituent atoms such as, without limitation, in the chirality of one or more stereocenters or related to the cis or trans configuration of a carbon-carbon or carbon-nitrogen double bond. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

“Alkyl” refers to monovalent saturated aliphatic hydrocarbyl groups having from 1 to 12 carbon atoms, preferably from 1 to 10 carbon atoms, and more preferably from 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—). Cₓ alkyl refers to an alkyl group having x number of carbon atoms.

“Alkenyl” refers to straight or branched monovalent hydrocarbyl groups having from 2 to 6 carbon atoms and preferably 2 to 4 carbon atoms and having at least 1 and preferably from 1 to 2 sites of vinyl (>C═C<) unsaturation. Such groups are exemplified, for example, by vinyl, allyl, and but-3-en-1-yl. Included within this term are the cis and trans isomers or mixtures of these isomers. Cₓ alkenyl refers to an alkenyl group having x number of carbon atoms.

“Alkynyl” refers to straight or branched monovalent hydrocarbyl groups having from 2 to 6 carbon atoms and preferably 2 to 3 carbon atoms and having at least 1 and preferably from 1 to 2 sites of acetylenic (—C≡C—) unsaturation. Examples of such alkynyl groups include acetylenyl (—C≡CH), and propargyl (—CH₂C≡CH). Cₓ alkynyl refers to an alkynyl group having x number of carbon atoms.

“Substituted alkyl” refers to an alkyl group having from 1 to 5, preferably 1 to 3, or more preferably 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, arylamino, substituted arylamino, heteroarylamino, substituted heteroarylamino, cycloalkylamino, substituted cycloalkylamino, heterocycloalkylamino, substituted heterocyclylamino, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, sulfonylamino, thioacyl, thiol, alkylthio, and substituted alkylthio, wherein said substituents are defined herein.

“Substituted alkenyl” refers to alkenyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, arylamino, substituted arylamino, heteroarylamino, substituted heteroarylamino, cycloalkylamino, substituted cycloalkylamino, heterocycloalkylamino, substituted heterocyclylamino, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, sulfonylamino, thioacyl, thiol, alkylthio, and substituted alkylthio, wherein said substituents are defined herein and with the proviso that any hydroxy or thiol substitution is not attached to a vinyl (unsaturated) carbon atom.

“Substituted alkynyl” refers to alkynyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, arylamino, substituted arylamino, heteroarylamino, substituted heteroarylamino, cycloalkylamino, substituted cycloalkylamino, heterocycloalkylamino, substituted heterocyclylamino, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, sulfonylamino, thioacyl, thiol, alkylthio, and substituted alkylthio, wherein said substituents are defined herein and with the proviso that any hydroxyl or thiol substitution is not attached to an acetylenic carbon atom.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Substituted alkoxy” refers to the group —O-(substituted alkyl) wherein substituted alkyl is defined herein. Preferred substituted alkyl groups in —O-(substituted alkyl) include halogenated alkyl groups and particularly halogenated methyl groups such as trifluoromethyl, difluromethyl, fluoromethyl and the like.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, aryl-C(O)—, substituted aryl-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic and substituted heterocyclic are as defined herein. Acyl includes the “acetyl” group CH₃C(O)—.

“Acylamino” refers to the groups —NR³⁰C(O)alkyl, —NR³⁰C(O)substituted alkyl, —NR³⁰C(O)cycloalkyl, —NR³⁰C(O)substituted cycloalkyl, —NR³⁰C(O)alkenyl, —NR³⁰C(O)substituted alkenyl, alkoxy, substituted alkoxy-NR³⁰C(O)alkynyl, —NR³⁰C(O)substituted alkynyl, —NR³⁰C(O)aryl, —NR³⁰C(O)substituted aryl, —NR³⁰C(O)heteroaryl, —NR³⁰C(O)substituted heteroaryl, —NR³⁰C(O)heterocyclic, and —NR³⁰C(O)substituted heterocyclic wherein R³⁰ is hydrogen, alkyl, substituted alkyl, aryl, substituted aryl, heteroaryl, substituted heteroaryl, cycloalkyl, or substituted cycloalkyl; and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Amino” refers to the group —NH₂.

“Substituted amino” refers to the group —NR³¹R³² where R³¹ and R³² are independently selected from the group consisting of hydrogen, alkyl, substituted alkyl, alkenyl, substituted alkenyl, alkoxy, substituted alkoxy, alkynyl, substituted alkynyl, aryl, substituted aryl, cycloalkyl, substituted cycloalkyl, heteroaryl, substituted heteroaryl, heterocyclic, substituted heterocyclic, arylamino, substituted arylamino, heteroarylamino, substituted heteroarylamino, cycloalkylamino, substituted cycloalkylamino, heterocycloalkylamino, substituted heterocyclylamino, sulfonylamino, and substituted sulfonyl and wherein R³¹ and R³² are optionally joined, together with the nitrogen bound thereto to form a heterocyclic or substituted heterocyclic group, provided that R³¹ and R³² are both not hydrogen, and wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic, and substituted heterocyclic are as defined herein. When R³¹ is hydrogen and R³² is alkyl, the substituted amino group is sometimes referred to herein as alkylamino. When R³¹ and R³² are alkyl, the substituted amino group is sometimes referred to herein as dialkylamino. When referring to a monosubstituted amino, it is meant that either R³¹ or R³² is hydrogen but not both. When referring to a disubstituted amino, it is meant that neither R³¹ nor R³² are hydrogen.

“Aminocarbonyl” refers to the group —C(O)NR³³R³⁴ where R³³ and R³⁴ are independently selected from the group consisting of hydrogen, alkyl, substituted alkyl, alkenyl, substituted alkenyl, alkoxy, substituted alkoxy, alkynyl, substituted alkynyl, aryl, substituted aryl, cycloalkyl, substituted cycloalkyl heteroaryl, substituted heteroaryl, heterocyclic, and substituted heterocyclic and where R³³ and R³⁴ are optionally joined together with the nitrogen bound thereto to form a heterocyclic or substituted heterocyclic group, and wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminothiocarbonyl” refers to the group —C(S)NR³³R³⁴ where R³³ and R³⁴ are independently selected from the group consisting of hydrogen, alkyl, substituted alkyl, alkenyl, substituted alkenyl, alkoxy, substituted alkoxy, alkynyl, substituted alkynyl, aryl, substituted aryl, cycloalkyl, substituted cycloalkyl, heteroaryl, substituted heteroaryl, heterocyclic, and substituted heterocyclic and where R³³ and R³⁴ are optionally joined together with the nitrogen bound thereto to form a heterocyclic or substituted heterocyclic group, and wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminocarbonylamino” refers to the group —NR³⁰C(O)NR³³R³⁴ where R³⁰ is hydrogen, alkyl, substituted alkyl, aryl, substituted aryl, heteroaryl, substituted heteroaryl, cycloalkyl, or substituted cycloalkyl, and R³³ and R³⁴ are independently selected from the group consisting of hydrogen, alkyl, substituted alkyl, alkenyl, substituted alkenyl, alkoxy, substituted alkoxy, alkynyl, substituted alkynyl, aryl, substituted aryl, cycloalkyl, substituted cycloalkyl, heteroaryl, substituted heteroaryl, heterocyclic, and substituted heterocyclic and where R³³ and R³⁴ are optionally joined together with the nitrogen bound thereto to form a heterocyclic or substituted heterocyclic group, and wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminothiocarbonylamino” refers to the group —NR³⁰C(S)NR³³R³⁴ where R³⁰ is hydrogen, alkyl, substituted alkyl, aryl, substituted aryl, heteroaryl, substituted heteroaryl, cycloalkyl, or substituted cycloalkyl, and R³³ and R³⁴ are independently selected from the group consisting of hydrogen, alkyl, substituted alkyl, alkenyl, substituted alkenyl, alkoxy, substituted alkoxy, alkynyl, substituted alkynyl, aryl, substituted aryl, cycloalkyl, substituted cycloalkyl, heteroaryl, substituted heteroaryl, heterocyclic, and substituted heterocyclic and where R³³ and R³⁴ are optionally joined together with the nitrogen bound thereto to form a heterocyclic or substituted heterocyclic group, and wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminocarbonyloxy” refers to the group —O—C(O)NR³³R³⁴ where R³³ and R³⁴ are independently selected from the group consisting of hydrogen, alkyl, substituted alkyl, alkenyl, substituted alkenyl, alkoxy, substituted alkoxy, alkynyl, substituted alkynyl, aryl, substituted aryl, cycloalkyl, substituted cycloalkyl, heteroaryl, substituted heteroaryl, heterocyclic, and substituted heterocyclic and where R³³ and R³⁴ are optionally joined together with the nitrogen bound thereto to form a heterocyclic or substituted heterocyclic group, and wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminosulfonyl” refers to the group —SO₂NR³³R³⁴ where R³³ and R³⁴ are independently selected from the group consisting of hydrogen, alkyl, substituted alkyl, alkenyl, substituted alkenyl, alkoxy, substituted alkoxy, alkynyl, substituted alkynyl, aryl, substituted aryl, cycloalkyl, substituted cycloalkyl, heteroaryl, substituted heteroaryl, heterocyclic, and substituted heterocyclic and where R³³ and R³⁴ are optionally joined together with the nitrogen bound thereto to form a heterocyclic or substituted heterocyclic group, and wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminosulfonyloxy” refers to the group —O—SO₂NR³³R³⁴ where R³³ and R³⁴ are independently selected from the group consisting of hydrogen, alkyl, substituted alkyl, alkenyl, substituted alkenyl, alkoxy, substituted alkoxy, alkynyl, substituted alkynyl, aryl, substituted aryl, cycloalkyl, substituted cycloalkyl, heteroaryl, substituted heteroaryl, heterocyclic, and substituted heterocyclic and where R³³ and R³⁴ are optionally joined together with the nitrogen bound thereto to form a heterocyclic or substituted heterocyclic group, and wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminosulfonylamino” refers to the group —NR³⁰—SO₂NR³³R³⁴ where R³⁰ is hydrogen, alkyl, substituted alkyl, aryl, substituted aryl, heteroaryl, substituted heteroaryl, cycloalkyl, or substituted cycloalkyl, and R³³ and R³⁴ are independently selected from the group consisting of hydrogen, alkyl, substituted alkyl, alkenyl, substituted alkenyl, alkoxy, substituted alkoxy, alkynyl, substituted alkynyl, aryl, substituted aryl, cycloalkyl, substituted cycloalkyl, heteroaryl, substituted heteroaryl, heterocyclic, and substituted heterocyclic and where R³³ and R³⁴ are optionally joined together with the nitrogen bound thereto to form a heterocyclic or substituted heterocyclic group, and wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic and substituted heterocyclic are as defined herein.

“Amidino” refers to the group —C(═NR³⁵)NR³³R³⁴ where R″, R³⁴, and R³⁵ are independently selected from the group consisting of hydrogen, alkyl, substituted alkyl, alkenyl, substituted alkenyl, alkoxy, substituted alkoxy, alkynyl, substituted alkynyl, aryl, substituted aryl, cycloalkyl, substituted cycloalkyl, heteroaryl, substituted heteroaryl, heterocyclic, and substituted heterocyclic and where R³³ and R³⁴ are optionally joined together with the nitrogen bound thereto to form a heterocyclic or substituted heterocyclic group, and wherein alkyl, substituted alkyl, alkenyl, substituted alkenyl, alkoxy, substituted alkoxy, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aryl” or “Ar” refers to a monovalent aromatic carbocyclic group of from 6 to 14 carbon atoms having a single ring (e.g., phenyl (Ph)) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is at an aromatic carbon atom. Preferred aryl groups include phenyl and naphthyl.

“Substituted aryl” refers to aryl groups which are substituted with 1 to 5, preferably 1 to 3, or more preferably 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, arylamino, substituted arylamino, heteroarylamino, substituted heteroarylamino, cycloalkylamino, substituted cycloalkylamino, heterocycloalkylamino, substituted heterocyclylamino carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, sulfonylamino, thioacyl, thiol, alkylthio, and substituted alkylthio, wherein said substituents are defined herein.

“Aryloxy” refers to the group —O-aryl, where aryl is as defined herein, that includes, by way of example, phenoxy and naphthoxy.

“Substituted aryloxy” refers to the group —O-(substituted aryl) where substituted aryl is as defined herein.

“Arylthio” refers to the group —S-aryl, where aryl is as defined herein.

“Substituted arylthio” refers to the group —S-(substituted aryl), where substituted aryl is as defined herein.

“Acylamino” refers to the group —NR³⁷ (aryl), where aryl is as defined herein and R³⁷ is hydrogen, alkyl, or substituted alkyl.

“Substituted arylamino” refers to the group —NR³⁷ (substituted aryl), where R³⁷ is hydrogen, alkyl, or substituted alkyl where substituted aryl is as defined herein.

“Carbonyl” refers to the divalent group —C(O)— which is equivalent to —C(═O)—.

“Carboxy” or “carboxyl” refers to —COOH or salts thereof.

“Carboxyl ester” or “carboxy ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-heteroaryl, —C(O)O-substituted heteroaryl, —C(O)O-heterocyclic, and —C(O)O-substituted heterocyclic wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“(Carboxyl ester)amino” refers to the group —NR³⁰—C(O)O-alkyl, —NR³⁰—C(O)O-substituted alkyl, —NR³⁰—C(O)O-alkenyl, —NR³⁰—C(O)O-substituted alkenyl, —NR³⁰—C(O)O-alkynyl, —NR³⁰—C(O)O-substituted alkynyl, —NR³⁰—C(O)O-aryl, —NR³⁰—C(O)O-substituted aryl, —NR³⁰—C(O)O-cycloalkyl, —NR³⁰—C(O)O-substituted cycloalkyl, —NR³⁰—C(O)O-heteroaryl, —NR³⁰—C(O)O-substituted heteroaryl, —NR³⁰—C(O)O-heterocyclic, and —NR³⁰—C(O)O-substituted heterocyclic wherein R³⁰ is alkyl or hydrogen, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“(Carboxyl ester)oxy” refers to the group —O—C(O)O-alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-heteroaryl, —O—C(O)O-substituted heteroaryl, —O—C(O)O-heterocyclic, and —O—C(O)O-substituted heterocyclic wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Cyano” refers to the group —C≡N.

“Cycloalkyl” refers to saturated or unsaturated but nonaromatic cyclic alkyl groups of from 3 to 10 carbon atoms, preferably from 3 to 8 carbon atoms, and more preferably from 3 to 6 carbon atoms, having single or multiple cyclic rings including fused, bridged, and Spiro ring systems. Cₓ cycloalkyl refers to a cycloalkyl group having x number of ring carbon atoms. Examples of suitable cycloalkyl groups include, for instance, adamantyl, cyclopropyl, cyclobutyl, cyclopentyl, and cyclooctyl. One or more the rings can be aryl, heteroaryl, or heterocyclic provided that the point of attachment is through the non-aromatic, non-heterocyclic ring saturated carbocyclic ring. “Substituted cycloalkyl” refers to a cycloalkyl group having from 1 to 5 or preferably 1 to 3 substituents selected from the group consisting of oxo, thione, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Cycloalkyloxy” refers to —O-cycloalkyl.

“Substituted cycloalkyloxy” refers to —O-(substituted cycloalkyl).

“Cycloalkylamino” refers to the group —NR³⁷ (cycloalkyl) where R³⁷ is hydrogen, alkyl, or substituted alkyl.

“Substituted cycloalkylamino” refers to the group —NR³⁷ (substituted cycloalkyl) where R³⁷ is hydrogen, alkyl, or substituted alkyl and substituted cycloalkyl is as defined herein.

“Cycloalkylthio” refers to —S-cycloalkyl.

“Substituted cycloalkylthio” refers to —S-(substituted cycloalkyl).

“Guanidino” refers to the group —NHC(═NH)NH₂.

“Substituted guanidino” refers to —NR³⁶C(═NR³⁶)N(R³⁶)₂ where each R³⁶ is independently selected from the group consisting of hydrogen, alkyl, substituted alkyl, aryl, substituted aryl, heteroaryl, substituted heteroaryl, heterocyclic, and substituted heterocyclic and two R³⁶ groups attached to a common guanidino nitrogen atom are optionally joined together with the nitrogen bound thereto to form a heterocyclic or substituted heterocyclic group, provided that at least one R³⁶ is not hydrogen, and wherein said substituents are as defined herein.

“Halo” or “halogen” refers to fluoro, chloro, bromo and iodo and preferably is fluoro or chloro.

“Hydroxy” or “hydroxyl” refers to the group —OH.

“Heteroalkylene” refers to an alkylene group wherein one or more carbons is replaced with —O—, —S—, —SO₂—, —NR^{Q}—,

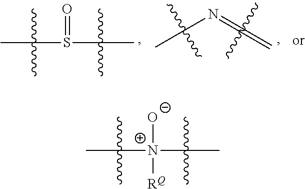

moieties where R^{Q} is H or C₁-C₆ alkyl. “Substituted heteroalkylene” refers to heteroalkynylene groups having from 1 to 3 substituents, and preferably 1 to 2 substituents, selected from the substituents disclosed for substituted alkylene.

“Heteroaryl” refers to an aromatic group of from 1 to 10 carbon atoms and 1 to 4 heteroatoms selected from the group consisting of oxygen, nitrogen and sulfur within the ring. Such heteroaryl groups can have a single ring (e.g., pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, or sulfonyl moieties. Preferred heteroaryls include 5 or 6 membered heteroaryls such as pyridinyl, pyrrolyl, thiophenyl, and furanyl. Other preferred heteroaryls include 9 or 10 membered heteroaryls, such as indolyl, quinolinyl, quinolonyl, isoquinolinyl, and isoquinolonyl.

“Substituted heteroaryl” refers to heteroaryl groups that are substituted with from 1 to 5, preferably 1 to 3, or more preferably 1 to 2 substituents selected from the group consisting of the same group of substituents defined for substituted aryl.

“Heteroaryloxy” refers to —O-heteroaryl.

“Substituted heteroaryloxy” refers to the group —O-(substituted heteroaryl).

“Heteroarylthio” refers to the group —S-heteroaryl.

“Substituted heteroarylthio” refers to the group —S-(substituted heteroaryl).

“Heteroarylamino” refers to the group —NR³⁷ (heteroaryl) where R³⁷ is hydrogen, alkyl, or substituted alkyl.

“Substituted heteroarylamino” refers to the group —NR³⁷ (substituted heteroaryl), where R³⁷ is hydrogen, alkyl, or substituted alkyl and substituted heteroaryl is defined as herein.

“Heterocycle” or “heterocyclic” or “heterocycloalkyl” or “heterocyclyl” refers to a saturated or partially saturated, but not aromatic, group having from 1 to 10 ring carbon atoms, preferably from 1 to 8 carbon atoms, and more preferably from 1 to 6 carbon atoms, and from 1 to 4 ring heteroatoms, preferably from 1 to 3 heteroatoms, and more preferably from 1 to 2 heteroatoms selected from the group consisting of nitrogen, sulfur, or oxygen. Cₓ heterocycloalkyl refers to a heterocycloalkyl group having x number of ring atoms including the ring heteroatoms. Heterocycle encompasses single ring or multiple condensed rings, including fused bridged and spiro ring systems. In fused ring systems, one or more the rings can be cycloalkyl, aryl or heteroaryl provided that the point of attachment is through the non-aromatic ring. In one embodiment, the nitrogen and/or sulfur atom(s) of the heterocyclic group are optionally oxidized to provide for the N-oxide, sulfinyl, sulfonyl moieties.

“Heterocyclylene” refers to a divalent saturated or partially saturated, but not aromatic, group having from 1 to 10 ring carbon atoms and from 1 to 4 ring heteroatoms selected from the group consisting of nitrogen, sulfur, or oxygen. “Substituted heterocyclylene” refers to heterocyclylene groups that are substituted with from 1 to 5 or preferably 1 to 3 of the same substituents as defined for substituted cycloalkyl.

“Substituted heterocyclic” or “substituted heterocycloalkyl” or “substituted heterocyclyl” refers to heterocyclyl groups that are substituted with from 1 to 5 or preferably 1 to 3 of the same substituents as defined for substituted cycloalkyl.

“Heterocyclyloxy” refers to the group —O-heterocycyl.

“Substituted heterocyclyloxy” refers to the group —O-(substituted heterocycyl).

“Heterocyclylthio” refers to the group —S-heterocycyl.

“Substituted heterocyclylthio” refers to the group —S-(substituted heterocycyl).

“Heterocyclylamino” refers to the group —NR³⁷ (heterocyclyl) where R³⁷ is hydrogen, alkyl, or substituted alkyl.

“Substituted heterocyclylamino” refers to the group —NR³⁷ (substituted heterocyclyl), where R³⁷ is hydrogen, alkyl, or substituted alkyl and substituted heterocyclyl is defined as herein.

Examples of heterocyclyl and heteroaryl include, but are not limited to, azetidinyl, pyrrolyl, imidazolyl, pyrazolyl, pyridyl, pyrazyl, pyrimidyl, pyridazyl, indolizyl, isoindolyl, indolyl, dihydroindolyl, indazolyl, purinyl, quinolizinyl, isoquinolinyl, quinolinyl, phthalazinyl, naphthylpyridinyl, quinoxalinyl, quinazolinyl, cinnolinyl, pteridinyl, carbazolyl, carbolinyl, phenanthridinyl, acridinyl, phenanthrolinyl, isothiazolyl, phenazinyl, isoxazolyl, phenoxazinyl, phenothiazinyl, imidazolidinyl, imidazolinyl, piperidinyl, piperazinyl, indolinyl, phthalimidyl, 1,2,3,4-tetrahydroisoquinolinyl, 4,5,6,7-tetrahydrobenzo[b]thiophenyl, thiazolyl, thiazolidinyl, thiophenyl, benzo[b]thiophenyl, morpholinyl, thiomorpholinyl (also referred to as thiamorpholinyl), 1,1-dioxothiomorpholinyl, piperidinyl, pyrrolidinyl, and tetrahydrofuranyl.

“Nitro” refers to the group —NO₂.

“Oxo” refers to the atom (═O) or (O).

“Spiro ring systems” refers to bicyclic ring systems that have a single ring carbon atom common to both rings.

“Sulfinyl” refers to the divalent group —S(O)— or —S(═O)—.

“Sulfonyl” refers to the divalent group —S(O)₂— or —S(═O)₂—.

“Substituted sulfonyl” refers to the group —SO₂-alkyl, —SO₂-substituted alkyl, —SO₂—OH, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein. Substituted sulfonyl includes groups such as methyl-SO₂—, phenyl-SO₂—, and 4-methylphenyl-SO₂—. Preferred substituted alkyl groups on the substituted alkyl-SO₂— include halogenated alkyl groups and particularly halogenated methyl groups such as trifluoromethyl, difluromethyl, fluoromethyl and the like.

“Substituted sulfinyl” refers to the group —SO-alkyl, —SO-substituted alkyl, —SO-alkenyl, —SO-substituted alkenyl, —SO-cycloalkyl, —SO-substituted cycloalkyl, —SO-aryl, —SO-substituted aryl, —SO-heteroaryl, —SO-substituted heteroaryl, —SO-heterocyclic, —SO-substituted heterocyclic, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein. Substituted sulfinyl includes groups such as methyl-SO—, phenyl-SO—, and 4-methylphenyl-SO—. Preferred substituted alkyl groups on the substituted alkyl-SO— include halogenated alkyl groups and particularly halogenated methyl groups such as trifluoromethyl, difluromethyl, fluoromethyl and the like.

“Sulfonyloxy” or “substituted sulfonyloxy” refers to the group —OSO₂-alkyl, —OSO₂-substituted alkyl, —OSO₂—OH, —OSO₂-alkenyl, —OSO₂-substituted alkenyl, —OSO₂-cycloalkyl, —OSO₂-substituted cycloalkyl, —OSO₂-aryl, —OSO₂-substituted aryl, —OSO₂-heteroaryl, —OSO₂-substituted heteroaryl, —OSO₂-heterocyclic, —OSO₂-substituted heterocyclic, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Sulfonylamino” refers to the group —NR³⁷ (substituted sulfonyl) where R³⁷ is hydrogen, alkyl, or substituted alkyl and substituted sulfonyl is as defined here.

“Thioacyl” refers to the groups H—C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, aryl-C(S)—, substituted aryl-C(S)—, heteroaryl-C(S)—, substituted heteroaryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Mercapto” or “thiol” refers to the group —SH.

“Formyl” refers to the group —C(O)H.

“Thiocarbonyl” refers to the divalent group —C(S)— which is equivalent to —C(═S)—.

“Thione” refers to the atom (═S).

“Alkylthio” refers to the group —S-alkyl wherein alkyl is as defined herein.

“Substituted alkylthio” refers to the group —S-(substituted alkyl) wherein substituted alkyl is as defined herein. Preferred substituted alkyl groups on —S-(substituted alkyl) include halogenated alkyl groups and particularly halogenated methyl groups such as trifluoromethyl, difluromethyl, fluoromethyl and the like.

“Vinyl” refers to unsaturated hydrocarbon radical —CH═CH₂, derived from ethylene.

The terms “optional” or “optionally” as used throughout the specification means that the subsequently described event or circumstance may but need not occur, and that the description includes instances where the event or circumstance occurs and instances in which it does not. For example, “the nitrogen atom is optionally oxidized to provide for the N-oxide (N→O) moiety” means that the nitrogen atom may but need not be oxidized, and the description includes situations where the nitrogen atom is not oxidized and situations where the nitrogen atom is oxidized.

The term “optionally substituted” refers to a substituted or unsubstituted group. The substituted group (e.g., the alkyl group in “substituted alkyl”) may be substituted with one or more substituents, such as e.g., 1, 2, 3, 4 or 5 substituents, which may be the same or different. Preferably, the substituents are selected from the functional groups provided herein. In certain more preferred embodiments, the substituents are selected from oxo, halo, —CN, NO₂, —CO₂R¹⁰⁰, —OR¹⁰⁰, +SR¹⁰⁰, —SOR¹⁰⁰, —SO₂R¹⁰⁰, —NR¹⁰¹R¹⁰², —CONR¹⁰¹R¹⁰², —SO₂NR¹⁰¹R¹⁰², C₁-C₆ alkyl, C₁-C₆ alkoxy, —CR¹⁰⁰═C(R¹⁰⁰)₂, CCR¹⁰⁰, C₃-C₁₀ cycloalkyl, C₄-C₁₀ heterocyclyl, C₆-C₁₄ aryl and C₅-C₁₂ heteroaryl, wherein each R¹⁰⁰ independently is hydrogen or C₁-C₈ alkyl; C₃-C₁₂ cycloalkyl; C₄-C₁₀ heterocyclyl; C₆-C₁₄ aryl; or C₂-C₁₂ heteroaryl; wherein each alkyl, cycloalkyl, heterocyclyl, aryl, or heteroaryl is optionally substituted with 1-3 halo, 1-3 C₁-C₆ alkyl, 1-3 C₁-C₆ haloalkyl or 1-3 C₁-C₆ alkoxy groups. More preferably, the substituents are selected from the group consisting of chloro, fluoro, —OCH₃, methyl, ethyl, iso-propyl, cyclopropyl, —OCF₃, —CF₃ and —OCHF₂.

R¹⁰¹ and R¹⁰² independently are hydrogen; C₁-C₈ alkyl, optionally substituted with —CO₂H or an ester thereof, C₁-C₆ alkoxy, oxo, —CR¹⁰³═C(R¹⁰³)₂, —CCR, C₃-C₁₀ cycloalkyl, C₃-C₁₀ heterocyclyl, C₆-C₁₄ aryl, or C₂-C₁₂ heteroaryl, wherein each R¹⁰³ independently is hydrogen or C₁-C₈ alkyl; C₃-C₁₂ cycloalkyl; C₄-C₁₀ heterocyclyl; C₆-C₁₄ aryl; or C₂-C₁₂ heteroaryl; wherein each cycloalkyl, heterocyclyl, aryl, or heteroaryl is optionally substituted with 1-3 alkyl groups or 1-3 halo groups, or R¹⁰¹ and R¹⁰² together with the nitrogen atom they are attached to form a 5-7 membered heterocycle.

Unless indicated otherwise, the nomenclature of substituents that are not explicitly defined herein are arrived at by naming the terminal portion of the functionality followed by the adjacent functionality toward the point of attachment. For example, the substituent “alkoxycarbonylalkyl” refers to the group (alkoxy)-C(O)-(alkyl)-.

It is understood that in all substituted groups defined above, polymers arrived at by defining substituents with further substituents to themselves (e.g., substituted aryl having a substituted aryl group as a substituent which is itself substituted with a substituted aryl group, etc.) are not intended for inclusion herein. In such cases, the maximum number of such substituents is three. That is to say that each of the above definitions is constrained by a limitation that, for example, substituted aryl groups are limited to -substituted aryl-(substituted aryl)-substituted aryl.

In some embodiments of a substituted moiety, the moiety is substituted with a group that may also be substituted with a further group, but the further group cannot be additionally substituted. For example, in some embodiments of “substituted alkyl”, the alkyl moiety is substituted with a group that may be further substituted (e.g., substituted alkoxy, substituted amino, substituted aryl, substituted aryloxy, substituted arylthio, substituted arylamino, substituted heteroarylamino, substituted cycloalkylamino, substituted heterocyclylamino, substituted cycloalkyl, substituted cycloalkyloxy, substituted cycloalkylthio, substituted guanidino, substituted heteroaryl, substituted heteroaryloxy, substituted heteroarylthio, substituted heterocyclic, substituted heterocyclyloxy, substituted heterocyclylthio, substituted sulfonyl, substituted alkylthio), but the substituted alkoxy, substituted amino, substituted aryl, substituted aryloxy, substituted arylthio, substituted arylamino, substituted heteroarylamino, substituted cycloalkylamino, substituted heterocyclylamino, substituted cycloalkyl, substituted cycloalkyloxy, substituted cycloalkylthio, substituted guanidino, substituted heteroaryl, substituted heteroaryloxy, substituted heteroarylthio, substituted heterocyclic, substituted heterocyclyloxy, substituted heterocyclylthio, substituted sulfonyl or substituted alkylthio on the alkyl moiety is not substituted with a moiety that is itself further substituted. Although “substituted alkyl” is provided as an example, such an embodiment is intended for each substituted moiety described herein.

In some embodiments of a substituted moiety, the moiety is substituted with a group that is not further substituted. Thus, in some embodiments, “substituted alkyl” is an alkyl moiety substituted with one or more, and in some aspects, 1 or 2 or 3 or 4 or 5 moieties independently selected from the group consisting of alkoxy, acyl, acylamino, acyloxy, amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, aryloxy, arylthio, arylamino, heteroarylamino, cycloalkylamino, heterocycloalkylamino, carboxyl, carboxyl ester, (carboxyl ester)amino, (carboxyl ester)oxy, cyano, cycloalkyl, cycloalkyloxy, cycloalkylthio, guanidino, halo, hydroxy, heteroaryl, heteroaryloxy, heteroarylthio, heterocyclic, heterocyclyloxy, heterocyclylthio, nitro, SO₃H, sulfonyloxy, sulfonylamino, thioacyl, thiol, and alkylthio. Although “substituted alkyl” is provided as an example, such an embodiment is intended for each substituted moiety described herein.

It is understood that the above definitions are not intended to include impermissible substitution patterns (e.g., methyl substituted with 4 fluoro groups). Such impermissible substitution patterns are well known to the skilled artisan.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination. All combinations of the embodiments pertaining to the chemical groups represented by the variables are specifically embraced by the present invention and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present invention and are disclosed herein just as if each and every such sub-combination of chemical groups was individually and explicitly disclosed herein.

### Compounds

In one aspect, provided is a compound of formula (I)

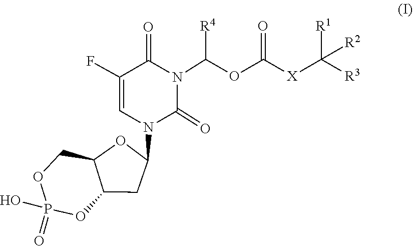

or a pharmaceutically acceptable salt thereof, wherein:
R¹ is H, optionally substituted C₁-C₆ alkyl, optionally substituted C₃-C₆ cycloalkyl, optionally substituted C₆-C₁₀ aryl, or optionally substituted 5- to 10-membered heteroaryl;
   or R¹ and one R⁵ group are taken together with the atoms to which they are attached to form an optionally substituted 5- to 7-membered heterocyclyl;
R² is H, optionally substituted C₁-C₆ alkyl, —OR⁵, or —N(R⁵)₂;
   or R¹ and R² are taken together with the carbon atom to which they are attached to form an optionally substituted 4- to 7-membered heterocyclyl or an optionally substituted C₃-C₆ cycloalkyl;
   R³ is H or optionally substituted C₁-C₆ alkyl;
   R⁴ is H, optionally substituted C₁-C₆ alkyl, or optionally substituted C₃-C₆ cycloalkyl;
   X is O, C(R⁶)₂, or a bond;
each R⁵ is independently H, optionally substituted C₁-C₆ alkyl, or optionally substituted C₃-C₆ cycloalkyl;
   or two R⁵ groups are taken together with the nitrogen atom to which they are attached to form an optionally substituted 5- to 7-membered heterocyclyl; and
each R⁶ is independently H, optionally substituted C₁-C₆ alkyl, or optionally substituted C₃-C₆ cycloalkyl;
   or two R⁶ groups are taken together with the carbon atom to which they are attached to form an optionally substituted C₃-C₆ cycloalkyl or an optionally substituted 3- to 7-membered heterocyclyl.

In some embodiments, X is O, C(R⁶)₂, or a bond. In some embodiments, X is O. In some embodiments, X is a bond. In some embodiments, X is C(R⁶)₂.

In some embodiments, each R⁶ is independently H, optionally substituted C₁-C₆ alkyl, or optionally substituted C₃-C₆ cycloalkyl; or two R⁶ groups are taken together with the carbon atom to which they are attached to form an optionally substituted C₃-C₆ cycloalkyl or an optionally substituted 3- to 7-membered heterocyclyl. In some embodiments, each R⁶ is independently H; C₁-C₆ alkyl optionally substituted with halogen, —OH, —CN, or —NH₂; or C₃-C₆ cycloalkyl optionally substituted with halogen, —OH, —CN, —NH₂, C₁-C₆ alkyl, or C₁-C₆ haloalkyl; or two R⁶ groups are taken together with the carbon atom to which they are attached to form a C₃-C₆ cycloalkyl or a 3- to 7-membered heterocyclyl, each of which is optionally substituted with halogen, —OH, —CN, —NH₂, C₁-C₆ alkyl, or C₁-C₆ haloalkyl. In some embodiments, each R⁶ is independently H or unsubstituted C₁-C₃ alkyl. In some embodiments, each R⁶ is independently H or —CH₃.

In some embodiments, each R⁶ is independently H, optionally substituted C₁-C₆ alkyl, or optionally substituted C₃-C₆ cycloalkyl. In some embodiments, each R⁶ is independently H; C₁-C₆ alkyl optionally substituted with halogen, —OH, —CN, or —NH₂; or C₃-C₆ cycloalkyl optionally substituted with halogen, —OH, —CN, —NH₂, C₁-C₆ alkyl, or C₁-C₆ haloalkyl. In some embodiments, two R⁶ groups are taken together with the carbon atom to which they are attached to form a C₃-C₆ cycloalkyl or a 3- to 7-membered heterocyclyl, each of which is optionally substituted with halogen, —OH, —CN, —NH₂, C₁-C₆ alkyl, or C₁-C₆ haloalkyl. In some embodiments, each R⁶ is independently H or unsubstituted C₁-C₃ alkyl. In some embodiments, each R⁶ is independently H or —CH₃.

In some embodiments, each R⁶ is independently optionally substituted C₁-C₆ alkyl. In some embodiments, each R⁶ is independently C₁-C₆ alkyl substituted with one, two, or three groups selected from the group consisting of halogen, —OH, —CN, and —NH₂. In some embodiments, each R⁶ is independently unsubstituted C₁-C₆ alkyl. In some embodiments, each R⁶ is independently C₁-C₃ alkyl optionally substituted with halogen, —OH, —CN, or —NH₂. In some embodiments, each R⁶ is independently C₁-C₃ alkyl, such as methyl, ethyl, n-propyl, or isopropyl, which is optionally substituted with halogen, —OH, —CN, or —NH₂. In some embodiments, each R⁶ is independently C₁-C₃ alkyl, such as methyl, ethyl, n-propyl, or isopropyl, which is substituted with one, two, or three groups selected from the group consisting of halogen, —OH, —CN, and —NH₂. In some embodiments, each R⁶ is independently unsubstituted C₁-C₃ alkyl, such as methyl, ethyl, n-propyl, or isopropyl. In some embodiments, both R⁶ groups are —CH₃.

In some embodiments, each R⁶ is independently optionally substituted C₃-C₆ cycloalkyl. In some embodiments, each R⁶ is independently C₃-C₆ cycloalkyl optionally substituted with halogen, —OH, —CN, —NH₂, C₁-C₆ alkyl, or C₁-C₆ haloalkyl. In some embodiments, each R⁶ is independently C₃-C₆ cycloalkyl is substituted with one, two, or three groups selected from the group consisting of halogen, —OH, —CN, —NH₂, C₁-C₆ alkyl, and C₁-C₆ haloalkyl. In some embodiments, each R⁶ is independently unsubstituted C₃-C₆ cycloalkyl.

In some embodiments, the two R⁶ groups are the same. In some embodiments, both R⁶ groups are H. In some embodiments, both R⁶ groups are C₁-C₆ alkyl substituted with one, two, or three groups selected from the group consisting of halogen, —OH, —CN, and —NH₂. In some embodiments, both R⁶ groups are C₁-C₃ alkyl substituted with one, two, or three groups selected from the group consisting of halogen, —OH, —CN, and —NH₂. In some embodiments, both R⁶ groups are unsubstituted C₁-C₃ alkyl. In some embodiments, both R⁶ groups are —CH₃.

In some embodiments, the two R⁶ groups are not the same. In some embodiments, at least one R⁶ group is C₁-C₆ alkyl optionally substituted with halogen, —OH, —CN, or —NH₂. In some embodiments, at least one R⁶ group is C₁-C₃ alkyl optionally substituted with halogen, —OH, —CN, or —NH₂. In some embodiments, at least one R⁶ group is unsubstituted C₁-C₃ alkyl, such as methyl, ethyl, n-propyl, or isopropyl. In some embodiments, at least one R⁶ group is C₃-C₆ cycloalkyl optionally substituted with halogen, —OH, —CN, —NH₂, C₁-C₆ alkyl, or C₁-C₆ haloalkyl. In some embodiments, at least one R⁶ group is unsubstituted C₃-C₆ cycloalkyl. In some embodiments, one R⁶ is H and the other R⁶ is optionally substituted C₁-C₆ alkyl or optionally substituted C₃-C₆ cycloalkyl.

In some embodiments, X is CH(C₁-C₆ alkyl), wherein the C₁-C₆ alkyl is optionally substituted with halogen, —OH, —CN, or —NH₂. In some embodiments, the C₁-C₆ alkyl is substituted with one, two, or three groups selected from the group consisting of halogen, —OH, —CN, and —NH₂. In some embodiments, X is CH(unsubstituted C₁-C₆ alkyl). In some embodiments, X is CH(C₁-C₃ alkyl), wherein the C₁-C₃ alkyl is optionally substituted with halogen, —OH, —CN, or —NH₂. In some embodiments, X is CH(C₁-C₃ alkyl), wherein the C₁-C₃ alkyl is substituted with one, two, or three groups selected from the group consisting of halogen, —OH, —CN, and —NH₂. In some embodiments, X is CH(methyl), CH(ethyl), CH(n-propyl), or CH(isopropyl), wherein the methyl, ethyl, n-propyl, and isopropyl are optionally substituted with halogen, —OH, —CN, or —NH₂. In some embodiments, X is CH(methyl), CH(ethyl), CH(n-propyl), or CH(isopropyl), wherein the methyl, ethyl, n-propyl, and isopropyl are substituted with one, two, or three groups selected from the group consisting of halogen, —OH, —CN, and —NH₂. In some embodiments, X is CH(methyl), CH(ethyl), CH(n-propyl), or CH(isopropyl). In some embodiments, X is CH(CH₃).

In some embodiments, X is CH(C₃-C₆ cycloalkyl), wherein the C₃-C₆ cycloalkyl is optionally substituted with halogen, —OH, —CN, —NH₂, C₁-C₆ alkyl, or C₁-C₆ haloalkyl. In some embodiments, the C₃-C₆ cycloalkyl is substituted with one, two, or three groups selected from the group consisting of halogen, —OH, —CN, —NH₂, C₁-C₆ alkyl, and C₁-C₆ haloalkyl. In some embodiments, the C₃-C₆ cycloalkyl is unsubstituted. In some embodiments, X is CH(cyclopropyl), CH(cyclobutyl), CH(cyclopentyl), or CH(cyclohexyl).

In some embodiments, two R⁶ groups are taken together with the carbon atom to which they are attached to form a C₃-C₆ cycloalkyl optionally substituted with halogen, —OH, —CN, —NH₂, C₁-C₆ alkyl, or C₁-C₆ haloalkyl. In some embodiments, the C₃-C₆ cycloalkyl is substituted with one, two, or three groups selected from the group consisting of halogen, —OH, —CN, —NH₂, C₁-C₆ alkyl, and C₁-C₆ haloalkyl. In some embodiments, the C₃-C₆ cycloalkyl is unsubstituted.

In some embodiments, two R⁶ groups are taken together with the carbon atom to which they are attached to form a 3- to 7-membered heterocyclyl optionally substituted with halogen, —OH, —CN, —NH₂, C₁-C₆ alkyl, or C₁-C₆ haloalkyl. In some embodiments, the 3- to 7-membered heterocyclyl is aziridinyl, oxiranyl, azetidinyl, oxetanyl, thietanyl, pyrrolidinyl, tetrahydrofuranyl, tetrahydrothiophenyl, piperidinyl, tetrahydropyranyl, thianyl, azepanyl, oxepanyl, thiepanyl, pyrazolidinyl, imidazolidinyl, dioxolanyl, oxazolidinyl, isoxazolidinyl, morpholinyl, or thiomorpholinyl, each of which is optionally substituted with halogen, —OH, —CN, —NH₂, C₁-C₆ alkyl, or C₁-C₆ haloalkyl. In some embodiments, the 3- to 7-membered heterocyclyl, including any variation detailed herein, is substituted with one, two, or three groups selected from the group consisting of halogen, —OH, —CN, —NH₂, C₁-C₆ alkyl, and C₁-C₆ haloalkyl. In some embodiments, the 3- to 7-membered heterocyclyl, including any variation detailed herein, is unsubstituted. In some embodiments, the 3- to 7-membered heterocyclyl contains one, two, or three heteroatoms selected from nitrogen, oxygen, sulfur, S(═O), and S(═O)₂ and is optionally substituted with halogen, —OH, —CN, —NH₂, C₁-C₆ alkyl, or C₁-C₆ haloalkyl. In some embodiments, the 3- to 7-membered heterocyclyl contains one or two nitrogen atoms. In some embodiments, the 3- to 7-membered heterocyclyl contains one nitrogen atom. In some embodiments, the 3- to 7-membered heterocyclyl contains one or two oxygen atoms. In some embodiments, the 3- to 7-membered heterocyclyl contains one oxygen atom. In some embodiments, the 3- to 7-membered heterocyclyl contains one or two sulfur atoms. In some embodiments, the 3- to 7-membered heterocyclyl contains one sulfur atom. In some embodiments, the 3- to 7-membered heterocyclyl contains one nitrogen atom and two oxygen atoms. In some embodiments, the 3- to 7-membered heterocyclyl contains two nitrogen atoms and one oxygen atom. In some embodiments, the 3- to 7-membered heterocyclyl contains one nitrogen atom and one oxygen atom. In some embodiments, the 3- to 7-membered heterocyclyl contains one nitrogen atom and two sulfur atoms. In some embodiments, the 3- to 7-membered heterocyclyl contains two nitrogen atoms and one sulfur atom. In some embodiments, the 3- to 7-membered heterocyclyl contains one nitrogen atom and one sulfur atom. In some embodiments, the 3- to 7-membered heterocyclyl contains one oxygen atom and two sulfur atoms. In some embodiments, the 3- to 7-membered heterocyclyl contains two oxygen atoms and one sulfur atom. In some embodiments, the 3- to 7-membered heterocyclyl contains one oxygen atom and one sulfur atom.

In some embodiments, R¹ is H, optionally substituted C₁-C₆ alkyl, optionally substituted C₃-C₆ cycloalkyl, optionally substituted C₆-C₁₀ aryl, or optionally substituted 5- to 10-membered heteroaryl. In some embodiments, R¹ and one R⁵ group are taken together with the atoms to which they are attached to form an optionally substituted 5- to 7-membered heterocyclyl. In some embodiments, R¹ and R² are taken together with the carbon atom to which they are attached to form an optionally substituted 4- to 7-membered heterocyclyl or an optionally substituted C₃-C₆ cycloalkyl. In some embodiments, R¹ is H; C₁-C₆ alkyl optionally substituted with halogen, —OH, —CN, or —NH₂; or C₃-C₆ cycloalkyl, C₆-C₁₀ aryl, or 5- to 10-membered heteroaryl, each of which is optionally substituted with halogen, —OH, —CN, —NH₂, C₁-C₆ alkyl, or C₁-C₆ haloalkyl. In some embodiments, R¹ is H or unsubstituted C₁-C₃ alkyl. In some embodiments, R¹ is H or —O-13. In some embodiments, R¹ and one R⁵ group are taken together with the atoms to which they are attached to form a 5- to 7-membered heterocyclyl optionally substituted with halogen, —OH, —NH₂, C₁-C₆ alkyl, or C₁-C₆ haloalkyl. In some embodiments, R¹ one R⁵ group are taken together with the atoms to which they are attached to form an unsubstituted 5- to 6-membered heterocyclyl. In some embodiments, R¹ and one R⁵ group are taken together with the atoms to which they are attached to form an unsubstituted 5-membered heterocyclyl. In some embodiments, R¹ and R² are taken together with the carbon atom to which they are attached to form a 4- to 7-membered heterocyclyl or C₃-C₆ cycloalkyl, each of which is optionally substituted with halogen, —OH, —CN, —NH₂, C₁-C₆ alkyl, or C₁-C₆ haloalkyl. In some embodiments, R¹ and R² are taken together with the carbon atom to which they are attached to form an unsubstituted 5- to 6-membered heterocyclyl. In some embodiments, R¹ and R² are taken together with the carbon atom to which they are attached to form an unsubstituted pyrrolidinyl.

In some embodiments, R¹ is H.

In some embodiments, R¹ is optionally substituted C₁-C₆ alkyl. In some embodiments, R¹ is C₁-C₆ alkyl optionally substituted with halogen, —OH, —CN, or —NH₂. In some embodiments, R¹ is C₁-C₆ alkyl substituted with one, two, or three groups selected from the group consisting of halogen, —OH, —CN, and —NH₂. In some embodiments, R¹ is unsubstituted

C₁-C₆ alkyl. In some embodiments, R¹ is C₁-C₃ alkyl, such as methyl, ethyl, n-propyl, or isopropyl, which is optionally substituted with halogen, —OH, —CN, or —NH₂. In some embodiments, R¹ is C₁-C₃ alkyl, such as methyl, ethyl, n-propyl, or isopropyl, which is substituted with one, two, or three groups selected from the group consisting of halogen, —OH, —CN, and —NH₂. In some embodiments, R¹ is unsubstituted C₁-C₃ alkyl, such as methyl, ethyl, n-propyl, or isopropyl. In some embodiments, R¹ is —CH₃.

In some embodiments, R¹ optionally substituted C₃-C₆ cycloalkyl. In some embodiments, R¹ is C₃-C₆ cycloalkyl optionally substituted with halogen, —OH, —CN, —NH₂, C₁-C₆ alkyl, or C₁-C₆ haloalkyl. In some embodiments, R¹ is C₃-C₆ cycloalkyl, such as cyclopropyl, cyclobutyl, cyclopentyl, or cyclohexyl, which is optionally substituted with halogen, —OH, —CN, —NH₂, C₁-C₆ alkyl, or C₁-C₆ haloalkyl. In some embodiments, R¹ is C₃-C₆ cycloalkyl, such as cyclopropyl, cyclobutyl, cyclopentyl, or cyclohexyl, which is substituted with one, two, or three groups selected from the group consisting of halogen, —OH, —CN, —NH₂, C₁-C₆ alkyl, and C₁-C₆ haloalkyl. In some embodiments, R¹ is unsubstituted C₃-C₆ cycloalkyl, such as cyclopropyl, cyclobutyl, cyclopentyl, or cyclohexyl.

In some embodiments, R¹ is optionally substituted C₆-C₁₀ aryl. In some embodiments, R¹ is C₆-C₁₀ aryl optionally substituted with halogen, —OH, —CN, —NH₂, C₁-C₆ alkyl, or C₁-C₆ haloalkyl. In some embodiments, R¹ is C₆-C₁₀ aryl, such as phenyl or naphthyl, which is optionally substituted with halogen, —OH, —CN, —NH₂, C₁-C₆ alkyl, or C₁-C₆ haloalkyl. In some embodiments, R¹ is C₆-C₁₀ aryl, such as phenyl or naphthyl, which is substituted with one, two, or three groups selected from the group consisting of halogen, —OH, —CN, —NH₂, C₁-C₆ alkyl, and C₁-C₆ haloalkyl. In some embodiments, R¹ is unsubstituted C₆-C₁₀ aryl, such as phenyl or naphthyl. In some embodiments, R¹ is phenyl substituted with one, two, or three groups selected from the group consisting of halogen, —OH, —CN, —NH₂, C₁-C₆ alkyl, and C₁-C₆ haloalkyl. In some embodiments, R¹ is unsubstituted phenyl.

In some embodiments, R¹ is optionally substituted 5- to 10-membered heteroaryl. In some embodiments, the 5- to 10-membered heteroaryl is optionally substituted with halogen, —OH, —CN, —NH₂, C₁-C₆ alkyl, or C₁-C₆ haloalkyl. In some embodiments, the 5- to 10-membered heteroaryl contains one, two or three heteroatoms selected from nitrogen, oxygen, sulfur, S(═O), and S(═O)₂ and is optionally substituted with halogen, —OH, —CN, —NH₂, C₁-C₆ alkyl, or C₁-C₆ haloalkyl. In some embodiments, R¹ is a 5- to 6-membered heteroaryl containing one, two or three heteroatoms selected from nitrogen, oxygen, sulfur, S(═O), and S(═O)₂ and optionally substituted with halogen, —OH, —CN, —NH₂, C₁-C₆ alkyl, or C₁-C₆ haloalkyl. In some embodiments, the 5- to 10-membered heteroaryl, such as a 5- to 6-membered heteroaryl, contains one or two nitrogen atoms. In some embodiments, the 5- to 10-membered heteroaryl contains one nitrogen atom. In some embodiments, the 5- to 10-membered heteroaryl contains one or two oxygen atoms. In some embodiments, the 5- to 10-membered heteroaryl contains one oxygen atom. In some embodiments, the 5- to 10-membered heteroaryl contains one or two sulfur atoms. In some embodiments, the 5- to 10-membered heteroaryl contains one sulfur atom. In some embodiments, the 5- to 10-membered heteroaryl contains one nitrogen atom and two oxygen atoms. In some embodiments, the 5- to 10-membered heteroaryl contains two nitrogen atoms and one oxygen atom. In some embodiments, the 5- to 10-membered heteroaryl contains one nitrogen atom and one oxygen atom. In some embodiments, the 5- to 10-membered heteroaryl contains one nitrogen atom and two sulfur atoms. In some embodiments, the 5- to 10-membered heteroaryl contains two nitrogen atoms and one sulfur atom. In some embodiments, the 5- to 10-membered heteroaryl contains one nitrogen atom and one sulfur atom. In some embodiments, the 5- to 10-membered heteroaryl contains one oxygen atom and two sulfur atoms. In some embodiments, the 5- to 10-membered heteroaryl contains two oxygen atoms and one sulfur atom. In some embodiments, the 5- to 10-membered heteroaryl contains one oxygen atom and one sulfur atom. In some embodiments, the 5- to 10-membered heteroaryl, such as pyrrolyl, pyrazolyl, imidazolyl, triazolyl, furanyl, thiophenyl, oxazolyl, thiazolyl, pyridinyl, pyridazinyl, pyrimidinyl, pyrazinyl, indolyl, indazolyl, benzimidazolyl, benzofuranyl, benzothiophenyl, quinolinyl, isoquinolinyl, quinoxalinyl, quinazolinyl, or naphthyridinyl, is optionally substituted with halogen, —OH, —CN, —NH₂, C₁-C₆ alkyl, or C₁-C₆ haloalkyl. In some embodiments, the 5- to 10-membered heteroaryl, including any variation detailed herein, is substituted with one, two, or three groups selected from the group consisting of halogen, —OH, —CN, —NH₂, C₁-C₆ alkyl, and C₁-C₆ haloalkyl. In some embodiments, the 5- to 10-membered heteroaryl, including any variation detailed herein, is unsubstituted.

In some embodiments, R¹ and one R⁵ group are taken together with the atoms to which they are attached to form an optionally substituted 5- to 7-membered heterocyclyl. In some embodiments, the 5- to 7-membered heterocyclyl is optionally substituted with halogen, —OH, —CN, —NH₂, C₁-C₆ alkyl, or C₁-C₆ haloalkyl. In some embodiments, the 5- to 7-membered heterocyclyl is substituted with one, two, or three groups selected from the group consisting of halogen, —OH, —CN, —NH₂, C₁-C₆ alkyl, and C₁-C₆ haloalkyl. In some embodiments, the 5- to 7-membered heterocyclyl is unsubstituted. In some embodiments, the 5- to 7-membered heterocyclyl contains one or two additional heteroatoms selected from nitrogen, oxygen, sulfur, S(═O), and S(═O)₂ and is optionally substituted with halogen, —OH, —CN, —NH₂, C₁-C₆ alkyl, or C₁-C₆ haloalkyl. In some embodiments, the additional heteroatoms are two nitrogen atoms. In some embodiments, the additional heteroatom is one nitrogen atom. In some embodiments, the additional heteroatoms are two oxygen atoms. In some embodiments, the additional heteroatom is one oxygen atom. In some embodiments, the additional heteroatoms are two sulfur atoms. In some embodiments, the additional heteroatom is one sulfur atom. In some embodiments, the additional heteroatoms are one nitrogen atom and one oxygen atom. In some embodiments, the additional heteroatoms are one nitrogen atom and one sulfur atom. In some embodiments, the additional heteroatoms are one oxygen atom and one sulfur atom. In some embodiments, the 5- to 7-membered heterocyclyl, such as

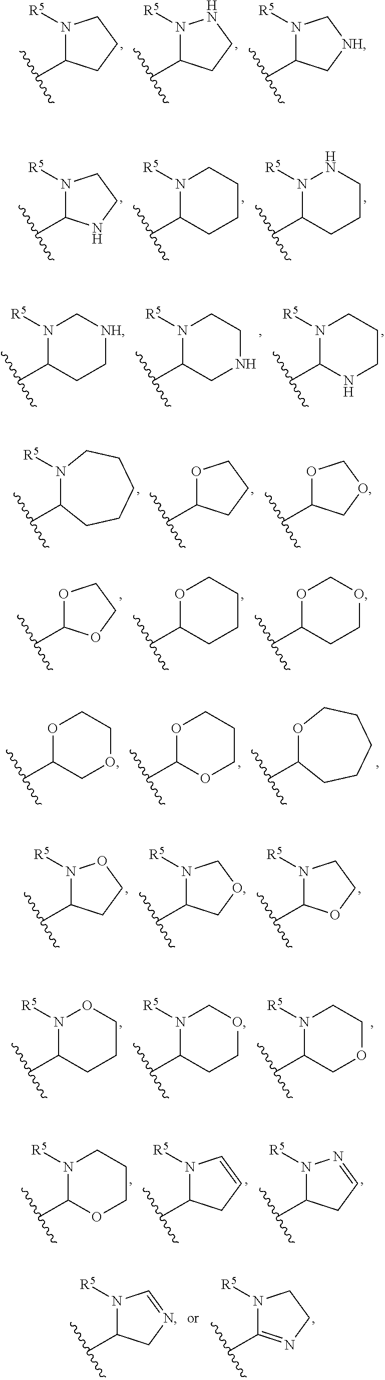

is optionally substituted with halogen, —OH, —CN, —NH₂, C₁-C₆ alkyl, or C₁-C₆ haloalkyl. In some embodiments, the 5- to 7-membered heterocyclyl, including any variation detailed herein, is substituted with one, two, or three groups selected from the group consisting of halogen, —OH, —CN, —NH₂, C₁-C₆ alkyl, and C₁-C₆ haloalkyl. In some embodiments, the 5- to 7-membered heterocyclyl, including any variation detailed herein, is unsubstituted. In some embodiments, the 5- to 7-membered heterocyclyl is a 5- to 6-membered heterocyclyl optionally substituted with halogen, —OH, —CN, —NH₂, C₁-C₆ alkyl, or C₁-C₆ haloalkyl. In some embodiments, the 5- to 7-membered heterocyclyl is unsubstituted 5- to 6-membered heterocyclyl. In some embodiments, the 5- to 7-membered heterocyclyl is a 5-membered heterocyclyl optionally substituted with halogen, —OH, —CN, —NH₂, C₁-C₆ alkyl, or C₁-C₆ haloalkyl. In some embodiments, the 5- to 7-membered heterocyclyl is unsubstituted 5-membered heterocyclyl. In some embodiments, the 5- to 7-membered heterocyclyl is pyrrolidin-2-yl optionally substituted with halogen, —OH, —CN, —NH₂, C₁-C₆ alkyl, or C₁-C₆ haloalkyl. In some embodiments, the 5- to 7-membered heterocyclyl is unsubstituted pyrrolidin-2-yl.

In some embodiments, R¹ and R² are taken together with the carbon atom to which they are attached to form an optionally substituted 4- to 7-membered heterocyclyl. In some embodiments, the 4- to 7-membered heterocyclyl is optionally substituted with halogen, —OH, —CN, —NH₂, C₁-C₆ alkyl, or C₁-C₆ haloalkyl. In some embodiments, the 4- to 7-membered heterocyclyl is substituted with one, two, or three groups selected from the group consisting of halogen, —OH, —CN, —NH₂, C₁-C₆ alkyl, and C₁-C₆ haloalkyl. In some embodiments, the 4- to 7-membered heterocyclyl is unsubstituted. In some embodiments, the 4- to 7-membered heterocyclyl contains one, two, or three heteroatoms selected from nitrogen, oxygen, sulfur, S(═O), and S(═O)₂ and is optionally substituted with halogen, —OH, —CN, —NH₂, C₁-C₆ alkyl, or C₁-C₆ haloalkyl. In some embodiments, the 4- to 7-membered heterocyclyl contains one or two nitrogen atoms. In some embodiments, the 4- to 7-membered heterocyclyl contains one nitrogen atom. In some embodiments, the 4- to 7-membered heterocyclyl contains one or two oxygen atoms. In some embodiments, the 4- to 7-membered heterocyclyl contains one oxygen atom. In some embodiments, the 4- to 7-membered heterocyclyl contains one or two sulfur atoms. In some embodiments, the 4- to 7-membered heterocyclyl contains one sulfur atom. In some embodiments, the 4- to 7-membered heterocyclyl contains one nitrogen atom and two oxygen atoms. In some embodiments, the 4- to 7-membered heterocyclyl contains two nitrogen atoms and one oxygen atom. In some embodiments, the 4- to 7-membered heterocyclyl contains one nitrogen atom and one oxygen atom. In some embodiments, the 4- to 7-membered heterocyclyl contains one nitrogen atom and two sulfur atoms. In some embodiments, the 4- to 7-membered heterocyclyl contains two nitrogen atoms and one sulfur atom. In some embodiments, the 4- to 7-membered heterocyclyl contains one nitrogen atom and one sulfur atom. In some embodiments, the 4- to 7-membered heterocyclyl contains one oxygen atom and two sulfur atoms. In some embodiments, the 4- to 7-membered heterocyclyl contains two oxygen atoms and one sulfur atom. In some embodiments, the 4- to 7-membered heterocyclyl contains one oxygen atom and one sulfur atom. In some embodiments, the 4- to 7-membered heterocyclyl, such as

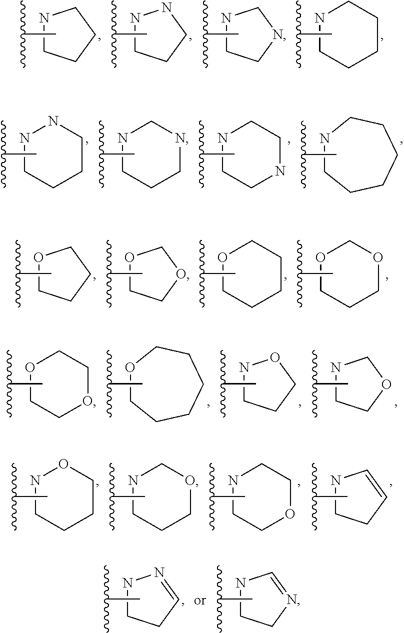

is optionally substituted with halogen, —OH, —CN, —NH₂, C₁-C₆ alkyl, or C₁-C₆ haloalkyl. In some embodiments, the 4- to 7-membered heterocyclyl, including any variation detailed herein, is substituted with one, two, or three groups selected from the group consisting of halogen, —OH, —CN, —NH₂, C₁-C₆ alkyl, and C₁-C₆ haloalkyl. In some embodiments, the 4- to 7-membered heterocyclyl, including any variation detailed herein, is unsubstituted. In some embodiments, the 4- to 7-membered heterocyclyl is a 5- to 6-membered heterocyclyl optionally substituted with halogen, —OH, —CN, —NH₂, C₁-C₆ alkyl, or C₁-C₆ haloalkyl. In some embodiments, the 4- to 7-membered heterocyclyl is unsubstituted 5- to 6-membered heterocyclyl. In some embodiments, the 4- to 7-membered heterocyclyl is a 5-membered heterocyclyl optionally substituted with halogen, —OH, —CN, —NH₂, C₁-C₆ alkyl, or C₁-C₆ haloalkyl. In some embodiments, the 4- to 7-membered heterocyclyl is unsubstituted 5-membered heterocyclyl. In some embodiments, the 4- to 7-membered heterocyclyl is pyrrolidinyl optionally substituted with halogen, —OH, —CN, —NH₂, C₁-C₆ alkyl, or C₁-C₆ haloalkyl. In some embodiments, the 4- to 7-membered heterocyclyl is unsubstituted pyrrolidinyl.

In some embodiments, R¹ and R² are taken together with the carbon atom to which they are attached to form an optionally substituted C₃-C₆ cycloalkyl. In some embodiments, the C₃-C₆ cycloalkyl is optionally substituted with halogen, —OH, —CN, —NH₂, C₁-C₆ alkyl, or C₁-C₆ haloalkyl. In some embodiments, the C₃-C₆ cycloalkyl is substituted with one, two, or three groups selected from the group consisting of halogen, —OH, —CN, —NH₂, C₁-C₆ alkyl, and C₁-C₆ haloalkyl. In some embodiments, the C₃-C₆ cycloalkyl is unsubstituted. In some embodiments, R¹ and R² are taken together with the carbon atom to which they are attached to form cyclopropyl, cyclobutyl, cyclopentyl, or cyclohexyl.

In some embodiments, R² is H, optionally substituted C₁-C₆ alkyl, —OR⁵, or —N(R⁵)₂; or R¹ and R² are taken together with the carbon atom to which they are attached to form an optionally substituted 4- to 7-membered heterocyclyl or an optionally substituted C₃-C₆ cycloalkyl. In some embodiments, R² is H; C₁-C₆ alkyl optionally substituted with halogen, —OH, —CN, or —NH₂; —OR⁵; or —N(R⁵)₂. In some embodiments, R² is H, unsubstituted C₁-C₃ alkyl, —O(unsubstituted C₁-C₆ alkyl), —OH, —NH₂, or —NH(unsubstituted C₁-C₆ alkyl). In some embodiments, R² is H, —CH₃, —OCH₃, —OH, —NH₂, or —N(H)CH₃.

In some embodiments, R² is H.

In some embodiments, R² is optionally substituted C₁-C₆ alkyl. In some embodiments, R² is C₁-C₆ alkyl optionally substituted with halogen, —OH, —CN, or —NH₂. In some embodiments, R² is C₁-C₆ alkyl substituted with one, two, or three groups selected from the group consisting of halogen, —OH, —CN, and —NH₂. In some embodiments, R² is unsubstituted C₁-C₆ alkyl. In some embodiments, R² is C₁-C₃ alkyl, such as methyl, ethyl, n-propyl, or isopropyl, which is optionally substituted with halogen, —OH, —CN, or —NH₂. In some embodiments, R² is C₁-C₃ alkyl, such as methyl, ethyl, n-propyl, or isopropyl, which is substituted with one, two, or three groups selected from the group consisting of halogen, —OH, —CN, and —NH₂. In some embodiments, R² is unsubstituted C₁-C₃ alkyl, such as methyl, ethyl, n-propyl, or isopropyl. In some embodiments, R² is —CH₃.

In some embodiments, R² is —OR⁵. In some embodiments, R² is —OH, —O(optionally substituted C₁-C₆ alkyl), or —O(optionally substituted C₃-C₆ cycloalkyl). In some embodiments, R² is —OH. In some embodiments, R² is —O(C₁-C₆ alkyl) optionally substituted with halogen, —OH, —CN, or —NH₂. In some embodiments, R² is —O(C₁-C₆ alkyl) substituted with one, two, or three groups selected from the group consisting of halogen, —OH, —CN, and —NH₂. In some embodiments, R² is —O(unsubstituted C₁-C₆ alkyl). In some embodiments, R² is —O(C₁-C₃ alkyl) optionally substituted with halogen, —OH, —CN, or —NH₂. In some embodiments, R² is —O(C₁-C₃ alkyl), such as —O(methyl), —O(ethyl), —O(n-propyl), or —O(isopropyl), which is optionally substituted with halogen, —OH, —CN, or —NH₂. In some embodiments, R² is —O(C₁-C₃ alkyl), such as —O(methyl), —O(ethyl), —O(n-propyl), or —O(isopropyl), which is substituted with one, two, or three groups selected from the group consisting of halogen, —OH, —CN, and —NH₂. In some embodiments, R² is —O(unsubstituted C₁-C₃ alkyl), such as —O(methyl), —O(ethyl), —O(n-propyl), or —O(isopropyl). In some embodiments, R² is —OCH₃. In some embodiments, R² is —O(C₃-C₆ cycloalkyl) optionally substituted with halogen, —OH, —CN, —NH₂, C₁-C₆ alkyl, or C₁-C₆ haloalkyl. In some embodiments, R² is —O(C₃-C₆ cycloalkyl), such as —O(cyclopropyl), —O(cyclobutyl), —O(cyclopentyl), or —O(cyclohexyl), which is optionally substituted with halogen, —OH, —CN, —NH₂, C₁-C₆ alkyl, or C₁-C₆ haloalkyl. In some embodiments, R² is —O(C₃-C₆ cycloalkyl), such as —O(cyclopropyl), —O(cyclobutyl), —O(cyclopentyl), or —O(cyclohexyl), which is substituted with one, two, or three groups selected from the group consisting of halogen, —OH, —CN, —NH₂, C₁-C₆ alkyl, and C₁-C₆ haloalkyl. In some embodiments, R² is —O(unsubstituted C₃-C₆ cycloalkyl), such as —O(cyclopropyl), —O(cyclobutyl), —O(cyclopentyl), or —O(cyclohexyl).

In some embodiments, R² is —N(R⁵)₂. In some embodiments, each R⁵ is independently H, optionally substituted C₁-C₆ alkyl, or optionally substituted C₃-C₆ cycloalkyl. In some embodiments, each R⁵ is independently C₁-C₆ alkyl optionally substituted with halogen, —OH, —CN, or —NH₂. In some embodiments, each R⁵ is independently unsubstituted C₁-C₆ alkyl. In some embodiments, each R⁵ is independently C₁-C₃ alkyl optionally substituted with halogen, —OH, —CN, or —NH₂. In some embodiments, each R⁵ is independently unsubstituted C₁-C₃ alkyl. In some embodiments, both R⁵ groups are —CH₃.

In some embodiments, R² is —N(R⁵)₂, and both R⁵ groups are the same. In some embodiments, R² is —NH₂. In some embodiments, R² is —N(CH₃)₂.

In some embodiments, R² is —N(R⁵)₂, and the two R⁵ groups are not the same. In some embodiments, at least one R⁵ is C₁-C₆ alkyl optionally substituted with halogen, —OH, —CN, or —NH₂. In some embodiments, at least one R⁵ is C₁-C₃ alkyl optionally substituted with halogen, —OH, —CN, or —NH₂. In some embodiments, at least one R⁵ is unsubstituted C₁-C₃ alkyl, such as methyl, ethyl, n-propyl, or isopropyl. In some embodiments, at least one R⁵ is C₃-C₆ cycloalkyl optionally substituted with halogen, —OH, —CN, —NH₂, C₁-C₆ alkyl, or C₁-C₆ haloalkyl. In some embodiments, at least one R⁵ is unsubstituted C₃-C₆ cycloalkyl. In some embodiments, one R⁵ is H and the other R⁵ is optionally substituted C₁-C₆ alkyl or optionally substituted C₃-C₆ cycloalkyl.

In some embodiments, R² is —NH(C₁-C₆ alkyl), wherein the C₁-C₆ alkyl is optionally substituted with halogen, —OH, —CN, or —NH₂. In some embodiments, R² is —NH(C₁-C₆ alkyl), wherein the C₁-C₆ alkyl is substituted with one, two, or three groups selected from the group consisting of halogen, —OH, —CN, and —NH₂. In some embodiments, R² is —NH(unsubstituted C₁-C₆ alkyl). In some embodiments, R² is —NH(C₁-C₃ alkyl), wherein the C₁-C₃ alkyl is optionally substituted with halogen, —OH, —CN, or —NH₂. In some embodiments, R² is —NH(C₁-C₃ alkyl), wherein the C₁-C₃ alkyl is substituted with one, two, or three groups selected from the group consisting of halogen, —OH, —CN, and —NH₂. In some embodiments, R² is —NH(methyl), —NH(ethyl), —NH(n-propyl), or —NH(isopropyl), wherein the methyl, ethyl, n-propyl, and isopropyl are optionally substituted with halogen, —OH, —CN, or —NH₂. In some embodiments, R² is —NH(methyl), —NH(ethyl), —NH(n-propyl), or —NH(isopropyl), wherein the methyl, ethyl, n-propyl, and isopropyl are substituted with one, two, or three groups selected from the group consisting of halogen, —OH, —CN, and —NH₂. In some embodiments, R² is —NH(methyl), —NH(ethyl), —NH(n-propyl), or —NH(isopropyl). In some embodiments, R² is —NH(CH₃).

In some embodiments, R² is —NH(C₃-C₆ cycloalkyl), wherein the C₃-C₆ cycloalkyl is optionally substituted with halogen, —OH, —CN, —NH₂, C₁-C₆ alkyl, or C₁-C₆ haloalkyl. In some embodiments, R² is —NH(C₃-C₆ cycloalkyl), wherein the C₃-C₆ cycloalkyl is substituted with one, two, or three groups selected from the group consisting of halogen, —OH, —CN, —NH₂, C₁-C₆ alkyl, and C₁-C₆ haloalkyl. In some embodiments, R² is —NH(unsubstituted C₃-C₆ cycloalkyl). In some embodiments, R² is —NH(cyclopropyl), —NH(cyclobutyl), —NH(cyclopentyl), or —NH(cyclohexyl).

In some embodiments, R² is —N(R⁵)₂ and two R⁵ groups are taken together with the nitrogen atom to which they are attached to form an optionally substituted 5- to 7-membered heterocyclyl. In some embodiments, the 5- to 7-membered heterocyclyl is optionally substituted with halogen, —OH, —CN, —NH₂, C₁-C₆ alkyl, or C₁-C₆ haloalkyl. In some embodiments, the 5- to 7-membered heterocyclyl is substituted with one, two, or three groups selected from the group consisting of halogen, —OH, —CN, —NH₂, C₁-C₆ alkyl, and C₁-C₆ haloalkyl. In some embodiments, the 5- to 7-membered heterocyclyl is unsubstituted. In some embodiments, the 5- to 7-membered heterocyclyl optionally contains one or two additional ring heteroatoms selected from the group consisting of nitrogen, oxygen, sulfur, S(═O), and S(═O)₂, and is optionally substituted with halogen, —OH, —CN, —NH₂, C₁-C₆ alkyl, or C₁-C₆ haloalkyl. In some embodiments, the 5- to 7-membered heterocyclyl contains two additional ring heteroatoms which are the same as each other. In some embodiments, the 5- to 7-membered heterocyclyl contains two additional ring heteroatoms which are different from each other. In some embodiments, the additional heteroatoms are two nitrogen atoms. In some embodiments, the additional heteroatom is one nitrogen atom. In some embodiments, the additional heteroatoms are two oxygen atoms. In some embodiments, the additional heteroatom is one oxygen atom. In some embodiments, the additional heteroatoms are two sulfur atoms. In some embodiments, the additional heteroatom is one sulfur atom. In some embodiments, the additional ring heteroatoms are one nitrogen atom and one oxygen atom. In some embodiments, the additional ring heteroatoms one nitrogen atom and one sulfur atom. In some embodiments, the additional ring heteroatoms are one oxygen atom and one sulfur atom. In some embodiments, the 5- to 7-membered heterocyclyl, such as

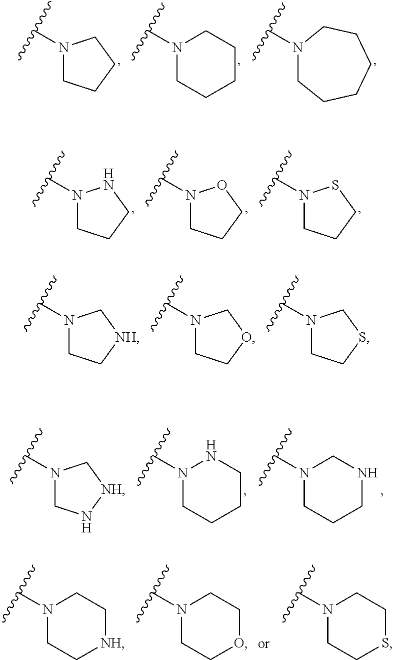

is optionally substituted with halogen, —OH, —CN, —NH₂, C₁-C₆ alkyl, or C₁-C₆ haloalkyl. In some embodiments, the 5- to 7-membered heterocyclyl, including any variation detailed herein, is substituted with one, two, or three groups selected from the group consisting of halogen, —OH, —CN, —NH₂, C₁-C₆ alkyl, and C₁-C₆ haloalkyl. In some embodiments, the 5- to 7-membered heterocyclyl, including any variation detailed herein, is unsubstituted. In some embodiments, the 5- to 7-membered heterocyclyl is a 5- to 6-membered heterocyclyl optionally substituted with halogen, —OH, —CN, —NH₂, C₁-C₆ alkyl, or C₁-C₆ haloalkyl. In some embodiments, the 5- to 7-membered heterocyclyl is an unsubstituted 5- to 6-membered heterocyclyl.

In some embodiments, R³ is H or optionally substituted C₁-C₆ alkyl. In some embodiments, R³ is H; or C₁-C₆ alkyl optionally substituted with halogen, —OH, —CN, or —NH₂. In some embodiments, R³ is H or unsubstituted C₁-C₃ alkyl. In some embodiments, R³ is H or —CH₃.

In some embodiments, R³ is H.

In some embodiments, R³ is optionally substituted C₁-C₆ alkyl. In some embodiments, R³ is C₁-C₆ alkyl optionally substituted with halogen, —OH, —CN, or —NH₂. In some embodiments, R³ is C₁-C₆ alkyl substituted with one, two, or three groups selected from the group consisting of halogen, —OH, —CN, and —NH₂. In some embodiments, R³ is unsubstituted C₁-C₆ alkyl. In some embodiments, R³ is C₁-C₃ alkyl, such as methyl, ethyl, n-propyl, or isopropyl, which is optionally substituted with halogen, —OH, —CN, or —NH₂. In some embodiments, R³ is C₁-C₃ alkyl, such as methyl, ethyl, n-propyl, or isopropyl, which is substituted with one, two, or three groups selected from the group consisting of halogen, —OH, —CN, and —NH₂. In some embodiments, R³ is unsubstituted C₁-C₃ alkyl, such as methyl, ethyl, n-propyl, or isopropyl. In some embodiments, R³ is —CH₃.

In some embodiments, R⁴ is H, optionally substituted C₁-C₆ alkyl, or optionally substituted C₃-C₆ cycloalkyl. In some embodiments, R⁴ is H; C₁-C₆ alkyl optionally substituted with halogen, —OH, —CN, or —NH₂; or C₃-C₆ cycloalkyl optionally substituted with halogen, —OH, —CN, —NH₂, C₁-C₆ alkyl, or C₁-C₆ haloalkyl. In some embodiments, R⁴ is H or unsubstituted C₁-C₃ alkyl. In some embodiments, R⁴ is H or —CH₃.

In some embodiments, R⁴ is H.

In some embodiments, R⁴ is optionally substituted C₁-C₆ alkyl. In some embodiments, R⁴ is C₁-C₆ alkyl optionally substituted with halogen, —OH, —CN, or —NH₂. In some embodiments, R⁴ is C₁-C₆ alkyl substituted with one, two, or three groups selected from the group consisting of halogen, —OH, —CN, and —NH₂. In some embodiments, R⁴ is unsubstituted C₁-C₆ alkyl. In some embodiments, R⁴ is C₁-C₃ alkyl, such as methyl, ethyl, n-propyl, or isopropyl, which is optionally substituted with halogen, —OH, —CN, or —NH₂. In some embodiments, R⁴ is C₁-C₃ alkyl, such as methyl, ethyl, n-propyl, or isopropyl, which is substituted with one, two, or three groups selected from the group consisting of halogen, —OH, —CN, and —NH₂. In some embodiments, R⁴ is unsubstituted C₁-C₃ alkyl, such as methyl, ethyl, n-propyl, or isopropyl. In some embodiments, R⁴ is —CH₃.

In some embodiments, R⁴ is optionally substituted C₃-C₆ cycloalkyl. In some embodiments, R⁴ is C₃-C₆ cycloalkyl optionally substituted with halogen, —OH, —CN, —NH₂, C₁-C₆ alkyl, or C₁-C₆ haloalkyl. In some embodiments, R⁴ is C₃-C₆ cycloalkyl, such as cyclopropyl, cyclobutyl, cyclopentyl, or cyclohexyl, which is optionally substituted with halogen, —OH, —CN, —NH₂, C₁-C₆ alkyl, or C₁-C₆ haloalkyl. In some embodiments, R⁴ is C₃-C₆ cycloalkyl, such as cyclopropyl, cyclobutyl, cyclopentyl, or cyclohexyl, which is substituted with one, two, or three groups selected from the group consisting of halogen, —OH, —CN, —NH₂, C₁-C₆ alkyl, and C₁-C₆ haloalkyl. In some embodiments, R⁴ is unsubstituted C₃-C₆ cycloalkyl, such as cyclopropyl, cyclobutyl, cyclopentyl, or cyclohexyl.

In some embodiments, each R⁵ is independently H, optionally substituted C₁-C₆ alkyl, or optionally substituted C₃-C₆ cycloalkyl; or two R⁵ groups are taken together with the nitrogen atom to which they are attached to form an optionally substituted 5- to 7-membered heterocyclyl; or R¹ and one R⁵ group are taken together with the atoms to which they are attached to form an optionally substituted 5- to 7-membered heterocyclyl. In some embodiments, R⁵ is H; C₁-C₆ alkyl optionally substituted with halogen, —OH, —CN, or —NH₂; or C₃-C₆ cycloalkyl optionally substituted with halogen, —OH, —CN, —NH₂, C₁-C₆ alkyl, or C₁-C₆ haloalkyl. In some embodiments, two R⁵ groups are taken together with the nitrogen atom to which they are attached to form a 5- to 7-membered heterocyclyl optionally substituted with halogen, —OH, —CN, —NH₂, C₁-C₆ alkyl, or C₁-C₆ haloalkyl, wherein the 5- to 7-membered heterocyclyl optionally contains 1-2 additional ring heteroatoms selected from the group consisting of O, N, S, S(═O), and S(═O)₂.

In some embodiments, at least one R⁵ is H. In some embodiments, both R⁵ groups are H. In some embodiments, R² is —OR⁵, and R⁵ is H.

In some embodiments, each R⁵ is independently optionally substituted C₁-C₆ alkyl. In some embodiments, each R⁵ is independently C₁-C₆ alkyl optionally substituted with halogen, —OH, —CN, or —NH₂. In some embodiments, each R⁵ is independently C₁-C₆ alkyl substituted with one, two, or three groups selected from the group consisting of halogen, —OH, —CN, and —NH₂. In some embodiments, each R⁵ is independently unsubstituted C₁-C₆ alkyl. In some embodiments, each R⁵ is independently C₁-C₃ alkyl, such as methyl, ethyl, n-propyl, or isopropyl, which is optionally substituted with halogen, —OH, —CN, or —NH₂. In some embodiments, each R⁵ is independently C₁-C₃ alkyl, such as methyl, ethyl, n-propyl, or isopropyl, which is substituted with one, two, or three groups selected from the group consisting of halogen, —OH, —CN, and —NH₂. In some embodiments, each R⁵ is independently unsubstituted C₁-C₃ alkyl, such as methyl, ethyl, n-propyl, or isopropyl. In some embodiments, at least one R⁵ is —CH₃. In some embodiments, both R⁵ groups are —CH₃. In some embodiments, R² is —OR⁵, and R⁵ is —CH₃.

In some embodiments, R⁵ is optionally substituted C₃-C₆ cycloalkyl. In some embodiments, R⁵ is C₃-C₆ cycloalkyl optionally substituted with halogen, —OH, —CN, —NH₂, C₁-C₆ alkyl, or C₁-C₆ haloalkyl. In some embodiments, R⁵ is C₃-C₆ cycloalkyl substituted with one, two, or three groups selected from the group consisting of halogen, —OH, —CN, —NH₂, C₁-C₆ alkyl, and C₁-C₆ haloalkyl. In some embodiments, R⁵ is unsubstituted C₃-C₆ cycloalkyl. In some embodiments, R⁵ is cyclopropyl, cyclobutyl, cyclopentyl, or cyclohexyl.

In some embodiments, the compound provided is of formula (II):

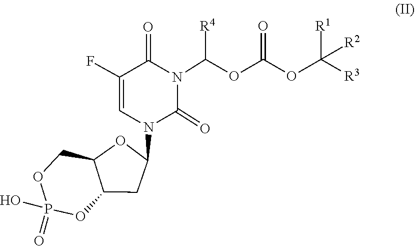

or a pharmaceutically acceptable salt thereof,
wherein R′, R², R³, and R⁴ are as defined herein for any embodiment or variation of a compound of formula (I). In some embodiments, R′, R², R³, and R⁴ are independently H or optionally substituted C₁-C₆ alkyl. In some embodiments, R¹ and R² are independently C₁-C₆ alkyl optionally substituted with halogen, —OH, —CN, or —NH₂. In some embodiments, R¹ and R² are independently unsubstituted C₁-C₃ alkyl. In some embodiments, R¹ and R² are both —CH₃. In some embodiments, R³ is H. In some embodiments, R⁴ is H. In some embodiments, R⁴ is unsubstituted C₁-C₃ alkyl. In some embodiments, R⁴ is —CH₃.

In some embodiments, the compound provided is of formula (III):

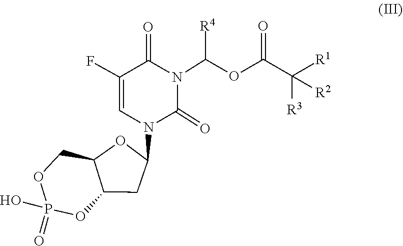

or a pharmaceutically acceptable salt thereof,
wherein R¹, R², R³, and R⁴ are as defined herein for any embodiment of a compound of formula (I). In some embodiments, R¹ is C₁-C₆ alkyl optionally substituted with halogen, —OH, —CN, or —NH₂. In some embodiments, R¹ is unsubstituted C₁-C₃ alkyl. In some embodiments, R¹ is —CH₃. In some embodiments, R² is —NH₂ or C₁-C₆ alkyl optionally substituted with halogen, —OH, —CN, or —NH₂. In some embodiments, R² is —NH₂. In some embodiments, R² is unsubstituted C₁-C₃ alkyl. In some embodiments, R² is —CH₃. In some embodiments, R³ is H or C₁-C₆ alkyl optionally substituted with halogen, —OH, —CN, or —NH₂. In some embodiments, R³ is H. In some embodiments, R³ is unsubstituted C₁-C₃ alkyl. In some embodiments, R³ is —CH₃. In some embodiments, R¹, R², and R³ are C₁-C₆ alkyl optionally substituted with halogen, —OH, —CN, or —NH₂. In some embodiments, R¹, R², and R³ are unsubstituted C₁-C₃ alkyl. In some embodiments, R¹, R², and R³ are —CH₃. In some embodiments, R¹ is C₁-C₆ alkyl optionally substituted with halogen, —OH, —CN, or —NH₂; R² is —NH₂; and R³ is H. In some embodiments, R¹ is —CH₃, R² is —NH₂, and R³ is H. In some embodiments, R¹ and R² are taken together with the carbon atom to which they are attached to form an optionally substituted 4- to 7-membered heterocyclyl. In some embodiments, R¹ and R² are taken together with the carbon atom to which they are attached to form an unsubstituted 5- to 6-membered heterocyclyl. In some embodiments, R¹ and R² are taken together with the carbon atom to which they are attached to form an unsubstituted pyrrolidinyl. In some embodiments, R⁴ is H.

In some embodiments, the compound provided is of formula (IV):

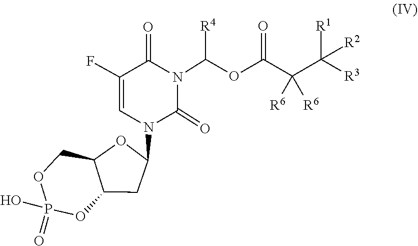

or a pharmaceutically acceptable salt thereof,
wherein R¹, R², R³, R⁴, and R⁶ are as defined herein for any embodiment of a compound of formula (I). In some embodiments, R¹, R², R³, R⁴, and R⁶ are independently H or optionally substituted C₁-C₆. In some embodiments, R¹, R², R³, and R⁴ are H. In some embodiments, each R⁶ is independently C₁-C₆ alkyl optionally substituted with halogen, —OH, —CN, or —NH₂. In some embodiments, each R⁶ is independently unsubstituted C₁-C₃ alkyl. In some embodiments, both R⁶ group are —CH₃.

In some embodiments, the compound provided is of formula (V):

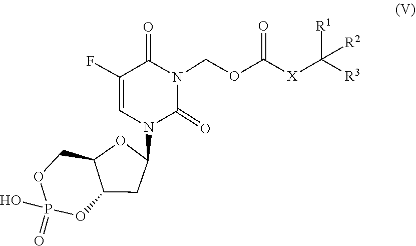

or a pharmaceutically acceptable salt thereof,
wherein R¹, R², R³, and X are as defined herein for any embodiment of a compound of formula (I). In some embodiments, R¹ is C₁-C₆ alkyl optionally substituted with halogen, —OH, —CN, or —NH₂. In some embodiments, R¹ is unsubstituted C₁-C₃ alkyl. In some embodiments, R¹ is —CH₃. In some embodiments, R¹ is H. In some embodiments, R² is —NH₂ or C₁-C₆ alkyl optionally substituted with halogen, —OH, —CN, or —NH₂. In some embodiments, R² is —NH₂. In some embodiments, R² is unsubstituted C₁-C₃ alkyl. In some embodiments, R² is —CH₃. In some embodiments, R³ is H or C₁-C₆ alkyl optionally substituted with halogen, —OH, —CN, or —NH₂. In some embodiments, R³ is H. In some embodiments, R³ is unsubstituted C₁-C₃ alkyl. In some embodiments, R³ is —CH₃. In some embodiments, R¹, R², and R³ are H. In some embodiments, R¹, R², and R³ are independently H or C₁-C₆ alkyl optionally substituted with halogen, —OH, —CN, or —NH₂. In some embodiments, R¹ and R² are taken together with the carbon atom to which they are attached to form an optionally substituted 4- to 7-membered heterocyclyl. In some embodiments, R¹ and R² are taken together with the carbon atom to which they are attached to form an unsubstituted 5- to 6-membered heterocyclyl. In some embodiments, R¹ and R² are taken together with the carbon atom to which they are attached to form an unsubstituted pyrrolidinyl. In some embodiment, X is O. In some embodiments, X is a bond. In some embodiments, X is C(R⁶)₂, wherein R⁶ is as defined herein for any embodiment of a compound of formula (I). In some embodiments, X is C(CH₃)₂.

In one aspect, provided is a compound of formula (I), or a pharmaceutically acceptable salt thereof, which has any one or more of the following structural features:
(I) X is:
   (i) 0;
   (ii) a bond; or
   (iii) C(C₁-C₆ alkyl)₂ optionally substituted with halogen or —OH,
(II) R¹ is:
   (iv) H; or
   (v) C₁-C₆ alkyl optionally substituted with halogen or —OH;
(III) R² is:
   (vi) H;
   (vii) C₁-C₆ alkyl optionally substituted with halogen or —OH; or
   (viii) NH₂;
(IV) R¹ and R² are taken together with the carbon atom to which they are attached to form a 4- to 7-membered heterocyclyl optionally substituted with halogen, —OH, or C₁-C₆ alkyl;
(V) R² is —N(R⁵)₂, wherein one R⁵ group and R¹ are taken together with the atoms to which they are attached to form a 5- to 7-membered heterocyclyl optionally substituted with halogen, —OH, or C₁-C₆ alkyl, and the other R⁵ group is H or C₁-C₆ alkyl optionally substituted with halogen or —OH;
(VI) R³ is:
   (ix) H; or
   (x) C₁-C₆ alkyl optionally substituted with halogen or —OH;
(VII) R⁴ is:
   (xi) H; or
   (xii) C₁-C₆ alkyl optionally substituted with halogen or —OH.
      In one variation, (I) applies. In one variation, (II) applies. In one variation, (III) applies. In one variation, (IV) applies. In one variation, (V) applies. In one variation, (VI) applies. In one variation, (VII) applies. In one aspect of this variation, (I), (II), (III), (VI), and (VII) apply. In another aspect of this variation, (I), (IV), (VI), and (VII) apply. In one variation, (I), (V), (VI), and (VII) apply. In one variation, (i), (v), (vii), (ix), and (xi) apply. In one variation, (i), (v), (vii), (ix), and (xii) apply. In one variation, (iii), (iv), (vi), (ix), and (xi) apply. In one variation, (ii), (v), (viii), (ix), and (xi) apply. In one variation, (ii), (V), (ix), and (xi) apply.

In the descriptions herein, it is understood that every description, variation, embodiment or aspect of a moiety may be combined with every description, variation, embodiment or aspect of other moieties the same as if each and every combination of descriptions is specifically and individually listed. For example, every description, variation, embodiment or aspect provided herein with respect to R⁴ of formula (I) may be combined with every description, variation, embodiment or aspect of R¹, R², R³, R⁵, R⁶, and X the same as if each and every combination were specifically and individually listed. It is also understood that all descriptions, variations, embodiments or aspects of formula (I), where applicable, apply equally to other formulae detailed herein, and are equally described, the same as if each and every description, variation, embodiment or aspect were separately and individually listed for all formulae. For example, all descriptions, variations, embodiments or aspects of formula (I), where applicable, apply equally to any of formulae as detailed herein, such as formulae (II), (III), (IV), and (V), and are equally described, the same as if each and every description, variation, embodiment or aspect were separately and individually listed for all formulae.

In some embodiments, provided is compound selected from compounds in Table 1, or pharmaceutically acceptable salt thereof. Although certain compounds described in Table 1 are presented as specific stereoisomers and/or in a non-stereochemical form, it is understood that any or all stereochemical forms, including any enantiomeric or diastereomeric forms, and any tautomers or other forms of any of the compounds of Table 1 are herein described.

TABLE 1

| Com- | |
|---|---|
| pound | |
| No. | Structure |
| 1 | 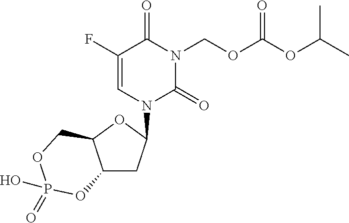 |
| 2 | 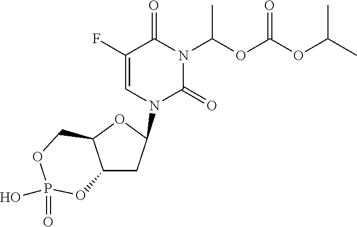 |
| 3 | 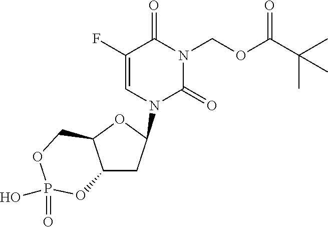 |
| 4 | 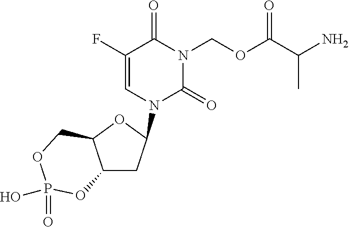 |
| 5 | 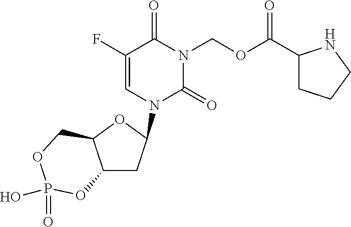 |

Also provided are salts of compounds referred to herein, such as pharmaceutically acceptable salts. The present disclosure also includes any or all of the stereochemical forms, including any enantiomeric or diastereomeric forms, and any tautomers or other forms of the compounds described. Thus, if a particular stereochemical form, such as a specific enantiomeric form or diastereomeric form, is depicted for a given compound, then it is understood that any or all stereochemical forms, including any enantiomeric or diastereomeric forms, and any tautomers or other forms of any of that same compound are herein described. Where tautomeric forms may be present for any of the compounds described herein, each and every tautomeric form is intended even though only one or some of the tautomeric forms may be explicitly depicted. The tautomeric forms specifically depicted may or may not be the predominant forms in solution or when used according to the methods described herein.

The disclosure also intends isotopically-labeled and/or isotopically-enriched forms of compounds described herein. The compounds herein may contain unnatural proportions of atomic isotopes at one or more of the atoms that constitute such compounds. In some embodiments, the compound is isotopically-labeled, such as an isotopically-labeled compound of the formula (I) or variations thereof described herein, where a fraction of one or more atoms are replaced by an isotope of the same element. Exemplary isotopes that can be incorporated into compounds described herein include isotopes of hydrogen, carbon, nitrogen, oxygen, phosphorus, sulfur, chlorine, such as ²H, ³H, ¹¹C, ¹³C, ¹⁴C ¹³N, ¹⁵O, ¹⁷O, ³²P, 3^{5S}, ¹⁸F, ³⁶Cl. Certain isotope labeled compounds (e.g. ³H and ¹⁴C) are useful in compound or substrate tissue distribution studies. Incorporation of heavier isotopes such as deuterium (²H) can afford certain therapeutic advantages resulting from greater metabolic stability, for example, increased in vivo half-life, or reduced dosage requirements and, hence may be preferred in some instances. Isotopically-labeled compounds described herein can generally be prepared by standard methods and techniques known to those skilled in the art or by procedures similar to those described in the accompanying Examples substituting appropriate isotopically-labeled reagents in place of the corresponding non-labeled reagent.

The disclosure also includes any or all metabolites of any of the compounds described. The metabolites may include any chemical species generated by a biotransformation of any of the compounds described, such as intermediates and products of metabolism of the compound, such as would be generated in vivo following administration to a human.

Solvates of a compound provided herein or a salt thereof are also contemplated. Solvates contain either stoichiometric or non-stoichiometric amounts of a solvent, and are often formed during the process of crystallization. Hydrates are formed when the solvent is water, or alcoholates are formed when the solvent is alcohol.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form. Unless otherwise stated, “substantially pure” intends a composition that contains no more than 35% impurity, wherein the impurity denotes a compound other than the compound comprising the majority of the composition or a salt thereof. In some embodiments, a composition of substantially pure compound or a salt thereof is provided wherein the composition contains no more than 25%, 20%, 15%, 10%, or 5% impurity. In some embodiments, a composition of substantially pure compound or a salt thereof is provided wherein the composition contains or no more than 3%, 2%, 1% or 0.5% impurity.

Articles of manufacture comprising a compound described herein, or a salt or solvate thereof, in a suitable container are provided. The container may be a vial, jar, ampoule, preloaded syringe, i.v. bag, and the like.

In some embodiments, the compounds detailed herein are orally bioavailable. In some embodiments, the compounds detailed herein are formulated for parenteral (e.g., intravenous) administration.

One or several compounds described herein can be used in the preparation of a medicament by combining the compound or compounds disclosed herein with a pharmacologically acceptable carrier, which are known in the art. Depending on the therapeutic form of the medication, the carrier may be in various forms. In one variation, the manufacture of a medicament is for use in any of the methods disclosed herein, e.g., for the treatment of liver cancer.

### Pharmaceutical Compositions and Formulations

Any of the prodrug compounds described herein may be formulated as a pharmaceutically acceptable composition.

Pharmaceutical compositions of any of the compounds detailed herein are embraced by this disclosure. Thus, the present disclosure includes pharmaceutical compositions comprising a compound as detailed herein, or a pharmaceutically acceptable salt thereof, and a pharmaceutically acceptable carrier or excipient. In one aspect, the pharmaceutically acceptable salt is an acid addition salt, such as a salt formed with an inorganic or organic acid. Pharmaceutical compositions may take a form suitable for oral, buccal, parenteral, nasal, topical or rectal administration or a form suitable for administration by inhalation.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form.

In one variation, the compounds herein are synthetic compounds prepared for administration to an individual. In another variation, compositions are provided containing a compound in substantially pure form. In another variation, the present disclosure embraces pharmaceutical compositions comprising a compound detailed herein and a pharmaceutically acceptable carrier. In another variation, methods of administering a compound are provided. The purified forms, pharmaceutical compositions and methods of administering the compounds are suitable for any compound or form thereof detailed herein.

A compound detailed herein, or a pharmaceutically acceptable salt thereof, may be formulated for any available delivery route, including an oral, mucosal (e.g., nasal, sublingual, vaginal, buccal or rectal), parenteral (e.g., intramuscular, subcutaneous or intravenous), topical or transdermal delivery form. A compound or salt thereof may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

A compound detailed herein, or a pharmaceutically acceptable salt thereof, can be used in the preparation of a formulation, such as a pharmaceutical formulation, by combining the compound or compounds, or a salt thereof, with a pharmaceutically acceptable carrier. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants. Formulations comprising the compound may also contain other substances which have valuable therapeutic properties. Pharmaceutical formulations may be prepared by known pharmaceutical methods. Suitable formulations can be found, e.g., in Remington's Pharmaceutical Sciences, Mack Publishing Company, Philadelphia, Pa., 20th ed. (2000), which is incorporated herein by reference.

A compound detailed herein, or a pharmaceutically acceptable salt thereof, may be administered to individuals in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid poly-ols, and so on. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants.

Any of the compounds described herein can be formulated in a tablet in any dosage form described, for example, a compound as described herein or a salt thereof can be formulated as a 10 mg tablet.

Compositions comprising a compound provided herein are also described. In one variation, the composition comprises a compound, or a pharmaceutically acceptable salt thereof, and a pharmaceutically acceptable carrier or excipient. In another variation, a composition of substantially pure compound is provided. In some embodiments, the composition is for use as a human or veterinary medicament. In some embodiments, the composition is for use in a method described herein. In some embodiments, the composition is for use in the treatment of a disease or disorder described herein.

Compositions formulated for co-administration of a compound provided herein and one or more additional pharmaceutical agents are also described. The co-administration can be simultaneous or sequential in any order. A compound provided herein may be formulated for co-administration with the one or more additional pharmaceutical agents in the same dosage form (e.g., single tablet or single i.v.) or separate dosage forms (e.g., two separate tablets, two separate i.v., or one tablet and one i.v.). Furthermore, co-administration can be, for example, 1) concurrent delivery, through the same route of delivery (e.g., tablet or i.v.), 2) sequential delivery on the same day, through the same route or different routes of delivery, or 3) delivery on different days, through the same route or different routes of delivery.

In one variation, a compound provided herein is metabolized to release a therapeutically effective amount of 5-FU and/or one or more 5-FU metabolites, such as 5-FdUMP. In some embodiments, the therapeutically effective amount of 5-FU and/or one or more 5-FU metabolites, such as 5-FdUMP, is effective in treating cancer. In some embodiments, the amount of 5-FU and/or one or more 5-FU metabolites, such as 5-FdUMP, in the bloodstream is effective in treating cancer. In one variation, a compound provided herein is metabolized to release one or more metabolites in an amount effective in treating cancer.

### Methods of Use

A prodrug is a pharmacologically inactive compound that is metabolized to a therapeutically active agent by one or more metabolic biotransformations. These metabolic biotransformations can occur when the prodrug is administered to a subject or cell. Metabolic processes include acid- or base-catalyzed chemical reaction(s) and enzyme-catalyzed chemical reaction(s). Embodiments are described herein wherein the therapeutically active compound is 5-FU, a metabolite of 5-FU (such as 5-FdUMP), and/or additional metabolites of the prodrugs described herein.

Compounds detailed herein are prodrugs of 5-FdUMP, which may improve bioavailability and/or efficacy and/or may reduce adverse reactions, as compared to 5-FU, in addition to other advantageous properties. In some embodiments, the compounds disclosed herein reduce the occurrence of side effects which result from a treatment regimen based on 5-FU. For example, in some embodiments, the compounds disclosed herein reduce the occurrence of palmar-plantar erythrodysesthesia (i.e., hand-foot syndrome). In some embodiments, the compounds disclosed herein reduce bone marrow toxicity.

Compounds and compositions detailed herein, such as a pharmaceutical composition containing a compound of any formula provided herein, or a pharmaceutically acceptable salt thereof, and a pharmaceutically acceptable carrier or excipient, may be used in methods of administration and treatment as provided herein. The compounds and compositions may also be used in in vitro methods, such as in vitro methods of administering a compound or composition to cells for screening purposes and/or for conducting quality control assays.

Provided herein is a method of treating a disease or disorder in an individual in need thereof comprising administering a compound described herein or any embodiment, variation, or aspect thereof, or a pharmaceutically acceptable salt thereof. In some embodiments, the compound, pharmaceutically acceptable salt thereof, or composition is administered to the individual according to a dosage and/or method of administration described herein.

In some embodiments, compounds and compositions detailed herein can transform to metabolites that inhibit the activity of thymidylate synthase. For example, the compounds of the disclosure may be metabolized to 5-FdUMP, which is known to inhibit thymidylate synthase. Accordingly, in some embodiments, provided herein is a method of inhibiting thymidylate synthase in a cell or in an individual or patient in need thereof comprising administering an effective amount of a compound or composition of the disclosure to the cell, individual, or patient. In some embodiments, thymidylate synthase is inhibited by a metabolite of the compound. In some embodiments, thymidylate synthase is inhibited by 5-FdUMP.

In some embodiments, provided herein is a method for treating a condition mediated by thymidylate synthase activity comprising administering to a mammal in need of treatment an effective amount of a compound of formula (I) or any related formula such as formula (II), (III), (IV), or (V), or a pharmaceutically acceptable salt thereof. In some embodiments, the condition is cancer, such as a cancer disclosed herein. In some embodiments, thymidylate synthase is inhibited by a metabolite of the compound. In some embodiments, thymidylate synthase is inhibited by 5-FdUMP.

In some embodiments, provided is a method for treating cancer, comprising administering to a mammal in need thereof an effective amount of a compound of formula (I) or any related formula such as formula (II), (III), (IV), or (V), or a pharmaceutically acceptable salt thereof. In some embodiments, the cancer is liver, colorectal, anal, breast, gastrointestinal, skin, stomach, esophageal, or pancreatic cancer. In some embodiments, the cancer is liver cancer. In some embodiments, the cancer originated from the liver or spread to the liver.

In one aspect, provided herein is a method of treating cancer, wherein modulation of thymidylate synthase activity prevents, inhibits, or ameliorates the pathology and/or symptomology of the cancer, in a patient, comprising administering to the patient a therapeutically effective amount of a compound or composition provided herein. In one embodiment, provided herein is a method of treating cancer, wherein modulation of thymidylate synthase activity prevents the pathology and/or symptomology of the cancer, in a patient, comprising administering to the patient a therapeutically effective amount of a compound or composition provided herein. In one embodiment, provided herein is a method of treating cancer, wherein modulation of thymidylate synthase activity inhibits the pathology and/or symptomology of the cancer, in a patient, comprising administering to the patient a therapeutically effective amount of a compound or composition provided herein. In one embodiment, provided herein is a method of treating a disease, wherein modulation of thymidylate synthase activity ameliorates the pathology and/or symptomology of the cancer, in a patient, comprising administering to the patient a therapeutically effective amount of a compound or composition provided herein. In some embodiments, thymidylate synthase is inhibited by a metabolite of the compound. In some embodiments, thymidylate synthase is inhibited by 5-FdUMP.

In another aspect, provided herein is a method of delaying the onset and/or development of a cancer that is mediated by thymidylate synthase activity in a patient (such as a human) who is at risk for developing the cancer. It is appreciated that delayed development may encompass prevention in the event the individual or patient does not develop the cancer. In some embodiments, thymidylate synthase is inhibited by a metabolite of the compound. In some embodiments, thymidylate synthase is inhibited by 5-FdUMP.

In one aspect, provided herein is a method of delaying the onset and/or development of cancer in a patient in need thereof, comprising administering to the patient a therapeutically effective amount of a compound or composition provided herein. In some embodiments, the cancer is liver, colorectal, anal, breast, gastrointestinal, skin, stomach, esophageal, or pancreatic cancer. In some embodiments, the cancer originated from the liver or spread to the liver. In one aspect, provided herein is a method of delaying the onset and/or development of liver cancer in a patient in need thereof, comprising administering to the patient a therapeutically effective amount of a compound or composition provided herein. In one variation, provided herein is a method of delaying the onset and/or development of cancer that originated in the liver in a patient in need thereof, comprising administering to the patient a therapeutically effective amount of a compound or composition provided herein. In one variation, provided herein is a method of delaying the onset and/or development of cancer that spread to the liver in a patient in need thereof, comprising administering to the patient a therapeutically effective amount of a compound or composition provided herein.

In one aspect, provided herein is a compound of formula (I) or any variation thereof, or a pharmaceutically acceptable salt thereof, for use in therapy. In some embodiments, provided herein is a compound of formula (I) or any variation thereof, or a pharmaceutically acceptable salt thereof or pharmaceutical composition comprising such compound or a pharmaceutically acceptable salt thereof, for use in the treatment of cancer. In some embodiments, provided is a compound of formula (I) or any variation thereof, or a pharmaceutically acceptable salt thereof, or a pharmaceutical composition comprising such compound or a pharmaceutically acceptable salt thereof, for use in the treatment of liver cancer. In some embodiments, provided is a compound of formula (I) or any variation thereof, or a pharmaceutically acceptable salt thereof, or a pharmaceutical composition comprising such compound or a pharmaceutically acceptable salt thereof, for use in the treatment of a cancer which originates from the liver or spreads to the liver.

In another embodiment, provided herein is a compound of formula (I) or any variation thereof, or a pharmaceutically acceptable salt thereof, for use in the manufacture of a medicament for the treatment of cancer. In some embodiments, the medicament is for the treatment of liver cancer. In some embodiments, the medicament is for the treatment of a cancer which originates from the liver or spreads to the liver.

In some embodiments, the cancer is sensitive to treatment by 5-FU. In some embodiments, the cancer is resistant to treatment by 5-FU. In some embodiments, the individual was previously treated with 5-FU or a 5-FU prodrug. In some embodiments, the previously administered 5-FU prodrug is capecitabine.

In some embodiments, the individual or patient is a mammal. In some embodiments, the patient is a primate, dog, cat, rabbit, or rodent. In some embodiments, the patient is a primate. In some embodiments, the patient is a human. In some embodiments, the human is at least about or is about any of 18, 21, 30, 50, 60, 65, 70, 75, 80, or 85 years old. In some embodiments, the human is a child. In some embodiments, the human is less than about or about any of 21, 18, 15, 10, 5, 4, 3, 2, or 1 years old.

In some embodiments, the method further comprises administering one or more additional pharmaceutical agents. In some embodiments, the method further comprises administering radiation. In some embodiments, the method further comprises administering one or more additional pharmaceutical agents and radiation.

In some embodiments, the method further comprises administering an additional thymidylate synthase inhibitor. In some embodiments, the method further comprises administering an agent which enhances the potency of the prodrug of formula (I) or any variation or aspect described herein, or a pharmaceutically acceptable salt thereof, or a metabolite thereof. In some embodiments, the method further comprises administering leucovorin.

In some embodiments, the method further comprises administering a platinum-based agent. In some embodiments, the method further comprises administering oxaliplatin or cisplatin. In some embodiments, the method further comprises administering leucovorin and oxaliplatin.

In some embodiments, the method further comprises administering a topoisomerase I inhibitor. In some embodiments, the method further comprises administering irinotecan. In some embodiments, the method further comprises administering leucovorin and irinotecan.

In some embodiments, the method further comprises administering mitomycin and/or methotrexate. In some embodiments, the method further comprises administering mitomycin. In some embodiments, the method further comprises administering methotrexate.

In some embodiments, the method further comprises administering a taxane. In some embodiments, the method further comprises administering a taxane and a platinum-based agent. In some embodiments, the method further comprises administering docetaxel or paclitaxel.

In some embodiments, the method further comprises administering one or more additional pharmaceutical agents which are useful for treating liver cancer (Vallanueva, A. (2019) N. Engl. J. Med., 380:1450-62). In some embodiments, the method further comprises administering one or more additional pharmaceutical agents which are cabozantinib-S-malate, pembrolizumab, lenvatinib mesylate, sorafenib tosylate, nivolumab, regorafenib, or combinations thereof. In some embodiments, the method further comprises administering cabozantinib-S-malate. In some embodiments, the method further comprises administering pembrolizumab. In some embodiments, the method further comprises administering lenvatinib mesylate. In some embodiments, the method further comprises administering sorafenib tosylate. In some embodiments, the method further comprises administering nivolumab. In some embodiments, the method further comprises administering regorafenib. In some embodiments, the method further comprises administering ramucirumab.

### Dosing and Method of Administration

The dose of a compound described herein, or a stereoisomer, tautomer, solvate, or salt thereof, administered to an individual (such as a human) may vary with the particular compound or salt thereof, the method of administration, and the particular cancer, such as type and stage of cancer, being treated. In some embodiments, the amount of the compound, or a stereoisomer, tautomer, solvate, or salt thereof, is a therapeutically effective amount.

The compounds provided herein or a salt thereof may be administered to a patient via various routes, including, e.g., intravenous, intramuscular, subcutaneous, oral, and transdermal.

The effective amount of the compound may in one aspect be a dose of between about 0.01 and about 100 mg/kg. Effective amounts or doses of the compounds of the present disclosure may be ascertained by routine methods, such as modeling, dose escalation, or clinical trials, taking into account routine factors, e.g., the mode or route of administration or drug delivery, the pharmacokinetics of the agent, the severity and course of the disease to be treated, the subject's health status, condition, and weight. An exemplary dose is in the range of about from about 0.7 mg to 7 g daily, or about 7 mg to 350 mg daily, or about 350 mg to 1.75 g daily, or about 1.75 to 7 g daily.

Any of the methods provided herein may in one aspect comprise administering to an individual a pharmaceutical composition that contains an effective amount of a compound provided herein, or a stereoisomer, tautomer, solvate, or salt thereof, and a pharmaceutically acceptable excipient.

A compound or composition provided herein may be administered to an individual in accordance with an effective dosing regimen for a desired period of time or duration, such as at least about one month, at least about 2 months, at least about 3 months, at least about 6 months, or at least about 12 months or longer, which in some variations may be for the duration of the individual's life. In one variation, the compound is administered on a daily or intermittent schedule. The compound can be administered to an individual continuously (for example, at least once daily) over a period of time. The dosing frequency can also be less than once daily, e.g., about a once weekly dosing. The dosing frequency can be more than once daily, e.g., twice or three times daily. The dosing frequency can also be intermittent, including a ‘drug holiday’ (e.g., once daily dosing for 7 days followed by no doses for 7 days, repeated for any 14 day time period, such as about 2 months, about 4 months, about 6 months or more). Any of the dosing frequencies can employ any of the compounds described herein together with any of the dosages described herein.

### Articles of Manufacture and Kits

The present disclosure further provides articles of manufacture comprising a compound described herein or a salt thereof, a composition described herein, or one or more unit dosages described herein in suitable packaging. In certain embodiments, the article of manufacture is for use in any of the methods described herein. Suitable packaging is known in the art and includes, for example, vials, vessels, ampules, bottles, jars, flexible packaging and the like. An article of manufacture may further be sterilized and/or sealed.

The present disclosure further provides kits for carrying out the methods of the present disclosure, which comprises one or more compounds described herein or a composition comprising a compound described herein. The kits may employ any of the compounds disclosed herein. In one variation, the kit employs a compound described herein or pharmaceutically acceptable salt thereof. The kits may be used for any one or more of the uses described herein, and, accordingly, may contain instructions for the treatment of any disease or described herein, for example for the treatment of cancer, including liver, colorectal, anal, breast, gastrointestinal, skin, stomach, esophageal, and pancreatic cancer. In some embodiments, the cancer originated from the liver or spread to the liver.

The kits optionally further comprise a container comprising one or more additional pharmaceutical agents and which kits further comprise instructions on or in the package insert for treating the subject with an effective amount of the one or more additional pharmaceutical agents. The one or more additional pharmaceutical agents may be leucovorin, cabozantinib-S-malate, pembrolizumab, lenvatinib mesylate, sorafenib tosylate, nivolumab, or regorafenib.

Kits generally comprise suitable packaging. The kits may comprise one or more containers comprising any compound described herein. Each component (if there is more than one component) can be packaged in separate containers or some components can be combined in one container where cross-reactivity and shelf life permit.

The kits may be in unit dosage forms, bulk packages (e.g., multi-dose packages) or sub-unit doses. For example, kits may be provided that contain sufficient dosages of a compound as disclosed herein and/or an additional pharmaceutically active compound useful for a disease detailed herein to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present disclosure. The instructions included with the kit generally include information as to the components and their administration to an individual.

### General Synthetic Methods

The compounds of the present disclosure may be prepared by a number of processes as generally described below and more specifically in the Examples hereinafter (such as the schemes provided in the Examples below). In the following process descriptions, the symbols when used in the formulae depicted are to be understood to represent those groups described above in relation to the formulae herein.

The intermediates described in the following preparations may contain a number of nitrogen, hydroxy, and acid protecting groups such as esters. The variable protecting group may be the same or different in each occurrence depending on the particular reaction conditions and the particular transformations to be performed. The protection and deprotection conditions are well known to the skilled artisan and are described in the literature. See. e.g., Greene and Wuts, Protective Groups in Organic Synthesis, (T. Greene and P. Wuts, eds., 2d ed. 1991).

Certain stereochemical centers have been left unspecified and certain substituents have been eliminated in the following schemes for the sake of clarity and are not intended to limit the teaching of the schemes in any way. Furthermore, individual isomers, enantiomers, and diastereomers may be separated or resolved by one of ordinary skill in the art at any convenient point in the synthesis of compounds of the invention, by methods such as selective crystallization techniques or chiral chromatography (See for example, J. Jacques, et al., “Enantiomers, Racemates, and Resolutions”, John Wiley and Sons, Inc., 1981, and E. L. Eliel and S. H. Wilen, “Stereochemistry of Organic Compounds”, Wiley-Interscience, 1994).

The compounds of the present invention, or salts thereof, may be prepared by a variety of procedures known in the art, some of which are illustrated in the Examples below. The specific synthetic steps for each of the routes described may be combined in different ways, to prepare compounds of the invention, or salts thereof. The products of each step can be recovered by conventional methods well known in the art, including extraction, evaporation, precipitation, chromatography, filtration, trituration, and crystallization. The reagents and starting materials are readily available to one of ordinary skill in the art. Others may be made by standard techniques of organic and heterocyclic chemistry which are analogous to the syntheses of known structurally-similar compounds and the procedures described in the Examples which follow including any novel procedures.

Compounds of general formula 1C (i.e., a compound of formula (I) as a triethylamine salt) can be prepared according to Scheme 1, wherein R¹, R², R³, R⁴, and X are as defined for formula (I), or any applicable variations detailed herein, and LG is a suitable leaving group (e.g., Cl or I).

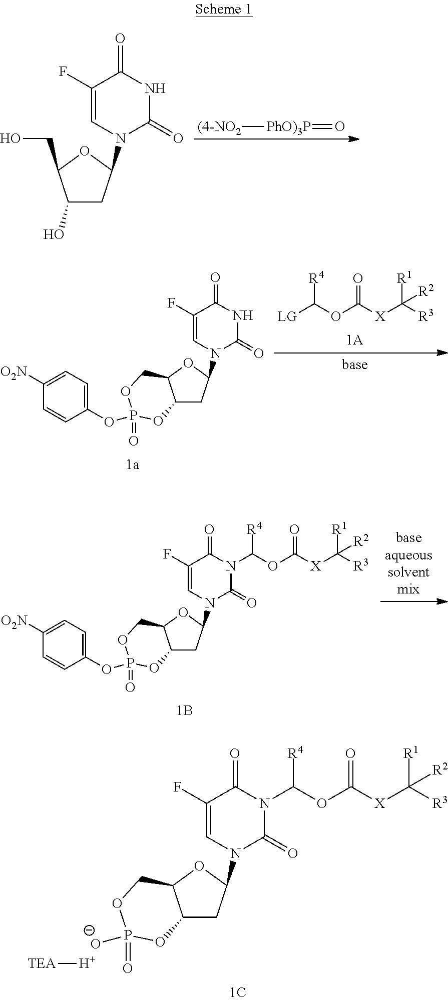

Coupling of 5-fluoro-2′-deoxy-uridine with tris(para-nitrophenoxy)phosphate, followed by cyclization, yields 1a, which can be alkylated with a compound of general formula 1A to give a compound of general formula 1B. Subsequent exposure to an appropriate base gives the compound of formula 1C.

Compounds of general formula (I) can be prepared according to Scheme 2, wherein R¹, R², R³, R⁴, and X are as defined for formula (I), or any applicable variations detailed herein, PG is a suitable protecting group (e.g., benzyl), and LG is a suitable leaving group (e.g., Cl or I).

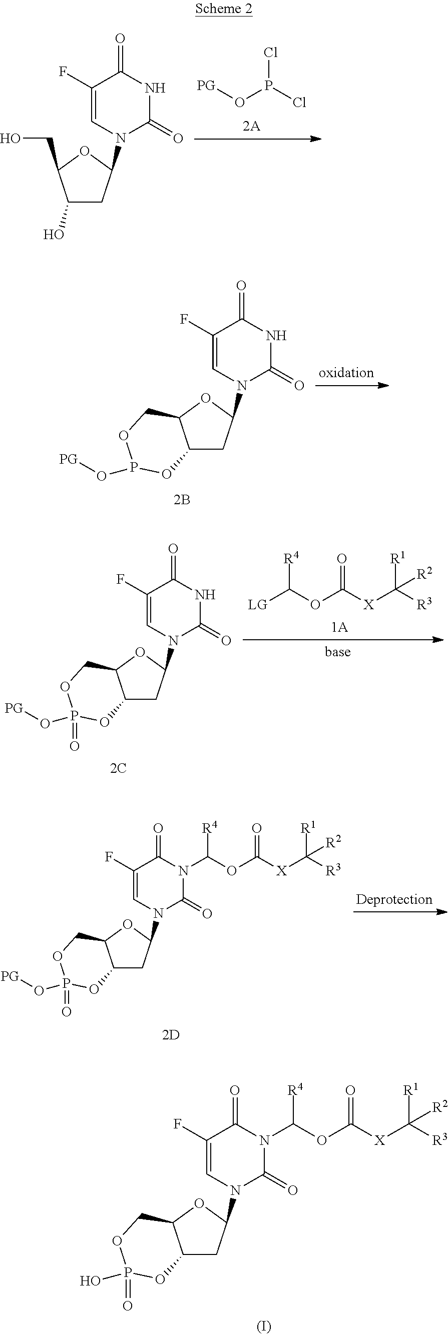

Coupling of 5-fluoro-2′-deoxy-uridine with a compound of general formula 2A, followed by cyclization, yields a compound of general formula 2B, which can be oxidized to give a compound of general formula 2C. Alkylation of the compound of general formula 2C via a compound of general formula 1A yields a compound of general formula 2D. Subsequent deprotection gives the compound of general formula (I).

### EXAMPLES

It is understood that the present disclosure has been made only by way of example, and that numerous changes in the combination and arrangement of parts can be resorted to by those skilled in the art without departing from the spirit and scope of present disclosure.

The chemical reactions in the Examples described can be readily adapted to prepare a number of other compounds disclosed herein, and alternative methods for preparing the compounds of this disclosure are deemed to be within the scope of this disclosure. For example, the synthesis of non-exemplified compounds according to the present disclosure can be successfully performed by modifications apparent to those skilled in the art, e.g., by appropriately protecting interfering groups, by utilizing other suitable reagents known in the art other than those described, or by making routine modifications of reaction conditions, reagents, and starting materials. Alternatively, other reactions disclosed herein or known in the art will be recognized as having applicability for preparing other compounds of the present disclosure.

Abbreviations used in the Examples include the following: DCM: dichloromethane; DBU: 1,8-diazabicyclo[5.4.0]undec-7-ene; EtOAc: ethyl acetate; ¹H NMR: Proton nuclear magnetic resonance; LCMS: liquid chromatography-mass spectrometry; MeOH: methanol or methyl alcohol; prep-HPLC: preparative high performance liquid chromatography; TEA: triethylamine; NMP: N-methyl-2-pyrrolidone; and THF: tetrahydrofuran.

### Example S1: Synthesis of (4aR,6R,7aS)-6-(5-fluoro-3-(((isopropoxycarbonyl)oxy)methyl)-2,4-dioxo-3,4-dihydropyrimidin-1(2H)-yl)tetrahydro-4H-furo[3,2-d][1,3,2]dioxaphosphinin-2-olate 2-oxide triethylamine salt (Compound No. 1—Tea Salt)

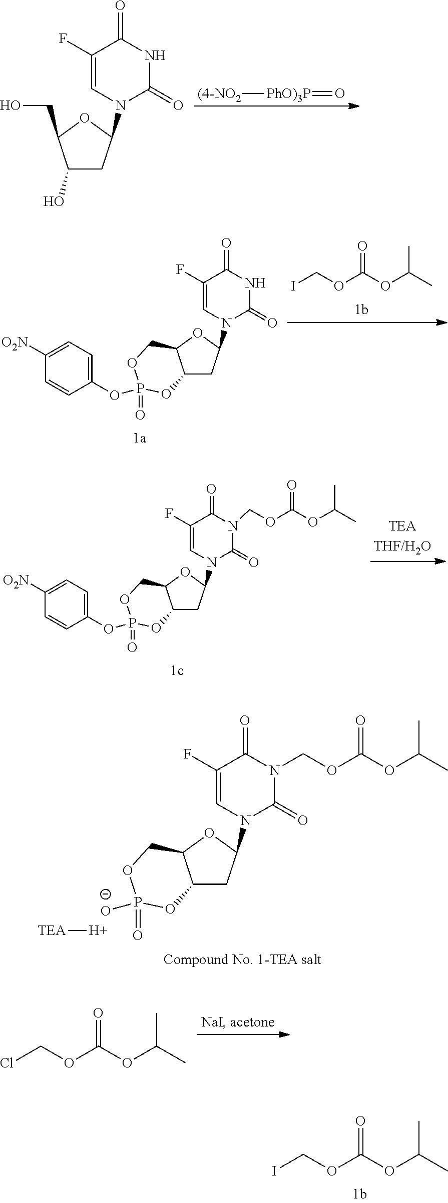

Synthesis of 1a. 5-Fluoro-2′-deoxy-uridine (5.0 g, 20.32 mmol) and tris(para-nitrophenoxy)phosphate (18.73 g, 40.65 mmol) were suspended in acetonitrile (200 mL). With stirring under nitrogen, DBU (12.13 mL, 81.3 mmol) was added carefully. The reaction was stirred for 30 min at room temperature. When complete (LCMS check), acetic acid (6 m, 100 mmol) was added. The solvents were removed by rotary evaporation to yield a reddish oil. The oil was washed with brine (4×150 mL). The brine washes were discarded. The remaining residue was dissolved in DCM and loaded onto silica gel. The products were eluded using EtOAc in hexanes (50 to 100%). The products are a mixture of two isomers at Phosphorus. Yield 3.1 g, 36%; M-H: 428.22

Synthesis of 1b. Chloromethyl isopropyl carbonate (6.42 mL, 48 mmol) was dissolved in acetone (150 mL). With stirring, NaI (7.91 g, 52.8 mmol) was added. The reaction mixture was heated at 55° C. for 4 h, after which the reaction was cooled to room temperature. The solid was filtered off and discarded. The reddish orange solution of the alkyl iodide in acetone was used in the next step without further purification assuming 100% yield.

Synthesis of 1c. Compound 1a (3.1 g, 7.23 mmol) and 1b (11.71 g, 48 mmol) in acetone (150 mL) were combined. Sodium carbonate (7.66 g, 72.3 mmol) was added at room temperature and stirred for 10 min. Potassium iodide (12.0 g, 72.3 mmol) was added and the reaction mixture was stirred for 4 h at room temperature. Water (15 mL) was added and stirring at room temperature was continued for ˜2 h while monitoring with LCMS. Acetic acid (10.84 mL) was added to quench the reaction and volatiles were removed in vacuo. The residue was partitioned between DCM (200 mL) and water (200 mL). The organic layer was washed with water (2×100 mL) and evaporated to dryness. The products were purified by flash chromatography on SiO₂ (eluent: EtOac in Hexanes 25 to 75%). Yield: 2.1 g, 53%.

Synthesis of (4aR,6R,7aS)-6-(5-fluoro-3-(((isopropoxycarbonyl)oxy)methyl)-2,4-dioxo-3,4-dihydropyrimidin-1(2H)-yl)tetrahydro-4H-furo[3,2-d][1,3,2]dioxaphosphinin-2-olate 2-oxide triethylamine salt (Compound No. 1—TEA salt). Compound 1c (91.63 g, 3.0 mmol) was dissolved in a THF/water (40 mL/40 mL). Triethylamine (3.3 mL, 24 mmol) was added and the reaction was heated at 55° C. for ˜8 h. Upon completion, the reaction was cooled to room temperature and formic acid (1.4 mL, 30 mmol) was added. Solvents were removed by rotary evaporation. The residue was dissolved in DCM (100 mL) and washed with brine (2×50 mL). The organic layer was evaporated to dryness. The residue was dissolved in water (100 mL) and was washed with EtOAc (2×50 mL). The product containing aqueous layer was saturated with sodium chloride and the pure product was extracted into DCM (4×100 mL). Evaporation and lyophilization from aqueous solution yielded Compound No. 1—TEA salt (1.18 g, 75%). Mass (m/z): 423.3 M-H. ¹H NMR [400 MHz] (MeOH-d4): δ=7.87 (d, J=6.8 Hz, 1H), 6.26 (d, J=7.6 Hz, 1H), 5.91 (s, 2H), 4.84 (m, 1H), 4.60 (m, 1H), 4.33 (m, 2H), 3.78 (m, 1H), 3.20 (q, J=7.6 Hz, 6H), 2.45 (m, 2H), 1.31 (t, J=7.6 Hz, 9H), 1.27 (d, J=6 Hz, 6H) ppm.

### Example S2: Synthesis of (5-fluoro-3-((4aR,6R,7a5)-2-hydroxy-2-oxidotetrahydro-4H-furo[3,2-d][1,3,2]dioxaphosphinin-6-yl)-2,6-dioxo-3,6-dihydropyrimidin-1(2H)-yl)methyl isopropyl carbonate (Compound No. 1—Free Base)

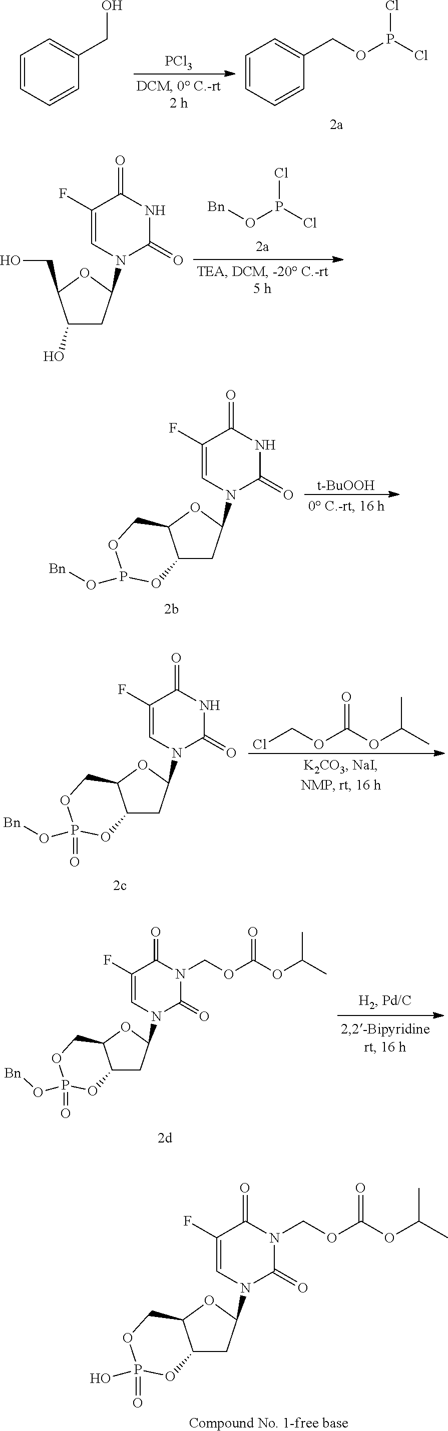

Synthesis of 2a. To a mixture of PCl₃ (10.1 mL, 116 mmol, 2.0 eq) in DCM (60 mL), cooled to 0° C., was added benzyl alcohol (6.0 g, 58 mmol, 1.0 eq) slowly over 15 min under nitrogen atmosphere. The mixture was stirred at room temperature for 2 h. The reaction was monitored by ¹H NMR. The reaction mixture was concentrated and the residue was distilled under reduced pressure to give dichlorobenzyloxyphosphite (2a) (4.2 g, 36%, 80-81° C., 0.2 mm Hg) as a colorless oil.

Synthesis of 2b. To a mixture of 5-fluoro-2′-deoxy-uridine (1 g, 4.1 mmol, 1.0 eq) in DCM (16 mL) was added TEA (1.65 g, 16.3 mmol, 4.0 eq) at room temperature, then cooled to −20° C., and dichlorobenzyloxyphosphite (2a) (1.1 g, 5.33 mmol, 1.3 eq) was added dropwise over 5 min. The mixture was stirred at −20° C. for 1 h. Then the mixture was slowly warmed to room temperature and stirred for 4 h. The reaction was monitored by TLC. Then the mixture was diluted with water and extracted with DCM. The organic layer was dried over anhydrous Na₂SO₄ and concentrated under reduced pressure. The residue was purified by silica gel chromatography to give the product 2b (380 mg, 24%).

Synthesis of 2c. To a mixture of 2b (870 mg, 2.28 mmol, 1.0 eq) in DCM was added tBuOOH (1.172 g, 9.11 mmol, 4.0 eq) at 0° C. The mixture was allowed to warm to room temperature and stirred at room temperature for 16 h under nitrogen atmosphere. The reaction was monitored by TLC. The resulting mixture was diluted with saturated aqueous Na₂SO₃ and extracted with DCM (3×100 mL). The organic phase was washed with brine, dried over Na₂SO₄ and concentrated under reduced pressure. The residue was purified by prep-HPLC to give the product 2c (260 mg, 28%).

Synthesis of 2d. To a solution of 2c (300 mg, 0.754 mmol, 1.0 eq) in NMP (5 mL) was added NaI (170 mg, 1.13 mol, 1.5 eq) at room temperature. Then chloromethyl isopropyl carbonate (172.5 mg, 1.13 mmol, 1.5 eq) and K2CO₃ (312.5 mg, 2.26 mmol, 3.0 eq) was added. The mixture was stirred at room temperature for 16 h. LCMS analysis of the reaction mixture showed full conversion to the desired product. Then the mixture was diluted with water and extracted with DCM. The organic layer was dried over anhydrous Na₂SO₄ and concentrated under reduced pressure. The residue was purified by prep-HPLC to afford the product (130 mg, 34%).

Synthesis of (5-fluoro-3-((4aR,6R,7aS)-2-hydroxy-2-oxidotetrahydro-4H-furo[3,2-d][1,3,2]dioxaphosphinin-6-yl)-2,6-dioxo-3,6-dihydropyrimidin-1(2H)-yl)methyl isopropyl carbonate (Compound No. 1—free base). To a solution of 2d (180 mg, 0.350 mmol, 1.0 eq) in MeOH (4 mL) was successively added 2,2′-bipyridine (27.3 mg, 0.175 mmol, 0.5 eq) and Pd/C (30 mg, 17% w/w) under nitrogen atmosphere. The mixture was charged with hydrogen 3 times and stirred at room temperature for 16 h. LCMS analysis of the reaction mixture showed full conversion to the desired product. The mixture was filtered through a pad of Celite and sintered funnel and the filtrate was concentrated under reduced pressure. The residue was purified by prep-HPLC to afford the product (Compound No. 1—free base) (130 mg, 87%). Mass (m/z): 423.3 M-H. ¹HNMR [400 MHz] (MeOH-d4): 8=7.87 (d, J=6.8 Hz, 1H), 6.24 (d, J=7.6 Hz, 1H), 5.91 (s, 2H), 4.84 (m, 1H), 4.61 (m, 1H), 4.33 (m, 2H), 3.79 (m, 1H), 2.46 (m, 2H), 1.27 (d, J=6 Hz, 6H) ppm.

### BIOLOGICAL EXAMPLES

### Example B1. Cytotoxicity Assay

Cytotoxicity of the test compounds were determined for multiple cell lines including gastric cancer cell lines NCI-N-87-luc and MKN45; liver cancer cell lines HepG2, Hep3B, and Huh7; pancreatic cell lines MIAPACA-2 and PANC-1; and colon cancer cell lines HT-29 and HCT-116.

Test compounds were prepared as 10 mM stock solutions. 5-FU, the control compound, was also prepared as a 10 mM stock solution. 45 μL of stock solution was transferred to a 384 polypropylene plate. The test and control compounds were then serially diluted 3 fold 10 times by transferring 15 μL compound solution into 30 μL DMSO by using TECAN (EVO200) liquid handler. 200 nL of each diluted compound solution was transferred into a separate well of a 384-well cell culture plate for each cell line assayed using an ECHO® 550 liquid transfer system. The cell culture plate was then placed in an incubator.

For each cell line assayed, cells were grown in a flask and then were harvested from the flask into cell culture medium. The cell number was counted and the cells were diluted with cell culture medium to the 2.5*10⁴ cells/mL. 40 μL of cell suspension was added into each well of the 384-well cell culture plate containing diluted compound solution. One plate for each cell line was prepared for extended treatment. A separate plate was prepared for Day 0 baseline detection.

The cell culture plates were covered with a lid, placed at room temperature for 30 min without shaking, and transferred into a 37° C. 5% CO₂ incubator for 72 h or 120 h. On the desired day, cytotoxicity of the compounds was detected using CellTiter Glo. The plates were removed from the incubators and allowed to equilibrate at room temperature for 15 min. CellTiter Glo reagents were thawed and allowed to equilibrate to room temperature. 40 μL of CellTiter-Glo reagent was added into each well to be detected (at 1:1 to culture medium). The plates were placed at room temperature for 30 min and then luminescence was detected using an EnSpire plate reader. The 50% cytotoxicity concentration (COO was then determined using Xlfit (v5.3.1.3). The calculated CC₅₀ for test compound (Compound No. 1—TEA salt) and 5-FU for each cell line are shown in Table 2. Compound No. 1 had dramatically lower CC₅₀ values compared to 5-FU for every cell line assayed.

TABLE 2

| | Compound No. 1 | 5-FU |
|---|---|---|
| Cell line | CC₅₀ (nM) | CC₅₀ (nM) |
| NCI-N-87-luc | 515 | 3,992 |
| MKN45 | 21 | 1,677 |
| HepG2 | 314 | 1,735 |
| Hep3B | 45 | 3,674 |
| Huh7 | 13 | 7,298 |
| MIAPACA-2 | 60 | 13,060 |
| PANC-1 | 706 | 8,255 |
| HT-29 | 15 | 14,821 |
| HCT-116 | 411 | 6,795 |

All publications, including patents, patent applications, and scientific articles, mentioned in this specification are herein incorporated by reference in their entirety for all purposes to the same extent as if each individual publication, including patent, patent application, or scientific article, were specifically and individually indicated to be incorporated by reference.

Although the foregoing invention has been described in some detail by way of illustration and example for purposes of clarity of understanding, it is apparent to those skilled in the art that certain minor changes and modifications will be practiced in light of the above teaching. Therefore, the description and examples should not be construed as limiting the scope of the invention.

1. A compound of formula (I) 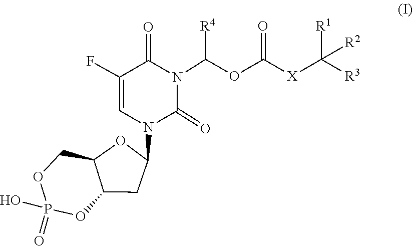
or a pharmaceutically acceptable salt thereof, wherein:
R¹ is H, optionally substituted C₁-C₆ alkyl, optionally substituted C₃-C₆ cycloalkyl, optionally substituted C₆-C₁₀ aryl, or optionally substituted 5- to 10-membered heteroaryl;
or R¹ and one R⁵ group are taken together with the atoms to which they are attached to form an optionally substituted 5- to 7-membered heterocyclyl;
R² is H, optionally substituted C₁-C₆ alkyl, —OR⁵, or —N(R⁵)₂;
or R¹ and R² are taken together with the carbon atom to which they are attached to form an optionally substituted 4- to 7-membered heterocyclyl or an optionally substituted C₃-C₆ cycloalkyl;
R³ is H or optionally substituted C₁-C₆ alkyl;
R⁴ is H, optionally substituted C₁-C₆ alkyl, or optionally substituted C₃-C₆ cycloalkyl;
X is O, C(R⁶)₂, or a bond;
each R⁵ is independently H, optionally substituted C₁-C₆ alkyl, or optionally substituted C₃-C₆ cycloalkyl;
or two R⁵ groups are taken together with the nitrogen atom to which they are attached to form an optionally substituted 5- to 7-membered heterocyclyl; and
each R⁶ is independently H, optionally substituted C₁-C₆ alkyl, or optionally substituted C₃-C₆ cycloalkyl;
or two R⁶ groups are taken together with the carbon atom to which they are attached to form an optionally substituted C₃-C₆ cycloalkyl or an optionally substituted 3- to 7-membered heterocyclyl.

2. The compound of claim 1, or a pharmaceutically acceptable salt thereof, wherein:
X is O.

3. The compound of claim 1, or a pharmaceutically acceptable salt thereof, wherein:
X is a bond.

4. The compound of claim 1, or a pharmaceutically acceptable salt thereof, wherein:
X is C(R⁶)₂.

5. The compound of claim 4, or a pharmaceutically acceptable salt thereof, wherein:
each R⁶ is independently H; C₁-C₆ alkyl optionally substituted with halogen, —OH, —CN, or —NH₂; or C₃-C₆ cycloalkyl optionally substituted with halogen, —OH, —CN, —NH₂, C₁-C₆ alkyl, or C₁-C₆ haloalkyl;
or two R⁶ groups are taken together with the carbon atom to which they are attached to form a C₃-C₆ cycloalkyl or a 3- to 7-membered heterocyclyl, each of which is optionally substituted with halogen, —OH, —CN, —NH₂, C₁-C₆ alkyl, or C₁-C₆ haloalkyl.

6. The compound of claim 5, or a pharmaceutically acceptable salt thereof, wherein:
each R⁶ is independently H or unsubstituted C₁-C₃ alkyl.

7. The compound of claim 6, or a pharmaceutically acceptable salt thereof, wherein:
each R⁶ is independently H or —CH₃.

8. The compound of any one of claims 1-7, or a pharmaceutically acceptable salt thereof, wherein:
R¹ is H; C₁-C₆ alkyl optionally substituted with halogen, —OH, —CN, or —NH₂; or C₃-C₆ cycloalkyl, C₆-C₁₀ aryl, or 5- to 10-membered heteroaryl, each of which is optionally substituted with halogen, —OH, —CN, —NH₂, C₁-C₆ alkyl, or C₁-C₆ haloalkyl,
or R¹ and one R⁵ group are taken together with the atoms to which they are attached to form a 5- to 7-membered heterocyclyl optionally substituted with halogen, —OH, —NH₂, C₁-C₆ alkyl, or C₁-C₆ haloalkyl.

9. The compound of claim 8, or a pharmaceutically acceptable salt thereof, wherein:
R¹ is H or unsubstituted C₁-C₃ alkyl,
or R¹ and one R⁵ group are taken together with the atoms to which they are attached to form an unsubstituted 5- to 6-membered heterocyclyl.

10. The compound of claim 9, or a pharmaceutically acceptable salt thereof, wherein:
R¹ is H or —CH₃,
or R¹ and one R⁵ group are taken together with the atoms to which they are attached to form an unsubstituted 5-membered heterocyclyl.

11. The compound of any one of claims 1-10, or a pharmaceutically acceptable salt thereof, wherein:
R² is H; C₁-C₆ alkyl optionally substituted with halogen, —OH, —CN, or —NH₂; —OR⁵; or —N(R⁵)₂.

12. The compound of claim 11, or a pharmaceutically acceptable salt thereof, wherein:
each R⁵ is independently H; C₁-C₆ alkyl optionally substituted with halogen, —OH, —CN, or —NH₂; or C₃-C₆ cycloalkyl optionally substituted with halogen, —OH, —CN, —NH₂, C₁-C₆ alkyl, or C₁-C₆ haloalkyl;
or two R⁵ groups are taken together with the nitrogen atom to which they are attached to form a 5- to 7-membered heterocyclyl optionally substituted with halogen, —OH, —CN, —NH₂, C₁-C₆ alkyl, or C₁-C₆ haloalkyl,
wherein the 5- to 7-membered heterocyclyl optionally contains 1-2 additional ring heteroatoms selected from the group consisting of O, N, S, S(═O), and S(═O)₂.

13. The compound of any one of claims 1-12, or a pharmaceutically acceptable salt thereof, wherein:
R² is H, unsubstituted C₁-C₃ alkyl, —O(unsubstituted C₁-C₆ alkyl), —OH, —NH₂, or —NH(unsubstituted C₁-C₆ alkyl).

14. The compound of any one of claims 1-13, or a pharmaceutically acceptable salt thereof, wherein:
R² is H, —CH₃, —OCH₃, —OH, —NH₂, or —N(H)CH₃.

15. The compound of any one of claims 1-7, or a pharmaceutically acceptable salt thereof, wherein:
R¹ and R² are taken together with the carbon atom to which they are attached to form a 4- to 7-membered heterocyclyl or C₃-C₆ cycloalkyl, each of which is optionally substituted with halogen, —OH, —CN, —NH₂, C₁-C₆ alkyl, or C₁-C₆ haloalkyl.

16. The compound of claim 15, or a pharmaceutically acceptable salt thereof, wherein:
R¹ and R² are taken together with the carbon atom to which they are attached to form an unsubstituted 5- to 6-membered heterocyclyl.

17. The compound of claim 16, or a pharmaceutically acceptable salt thereof, wherein the 5- to 6-membered heterocyclyl is unsubstituted pyrrolidinyl.

18. The compound of any one of claims 1-17, or a pharmaceutically acceptable salt thereof, wherein:
R³ is H; or C₁-C₆ alkyl optionally substituted with halogen, —OH, —CN, or —NH₂.

19. The compound of claim 18, or a pharmaceutically acceptable salt thereof, wherein:
R³ is H or unsubstituted C₁-C₃ alkyl.

20. The compound of claim 19, or a pharmaceutically acceptable salt thereof, wherein:
R³ is H or —CH₃.

21. The compound of any one of claims 1-20, or a pharmaceutically acceptable salt thereof, wherein:
R⁴ is H; C₁-C₆ alkyl optionally substituted with halogen, —OH, —CN, or —NH₂; or C₃-C₆ cycloalkyl optionally substituted with halogen, —OH, —CN, —NH₂, C₁-C₆ alkyl, or C₁-C₆ haloalkyl.

22. The compound of claim 21, or a pharmaceutically acceptable salt thereof, wherein:
R⁴ is H or unsubstituted C₁-C₃ alkyl.

23. The compound of claim 22, or a pharmaceutically acceptable salt thereof, wherein:
R⁴ is H or —CH₃.

24. A compound which is selected from the group consisting of: 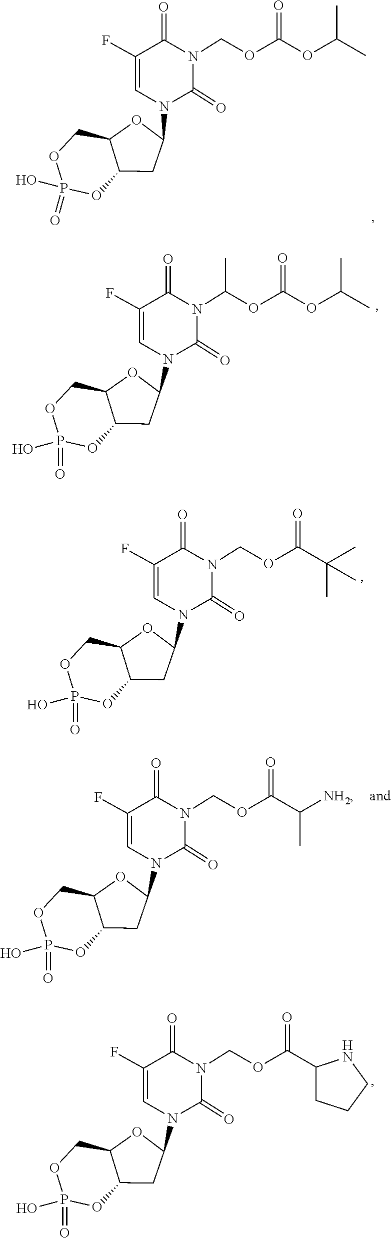
or a pharmaceutically acceptable salt thereof.

25. A pharmaceutical composition comprising the compound of any one of claims 1-24, or a pharmaceutically acceptable salt thereof, and at least one pharmaceutically acceptable carrier, diluent, and excipient.

26. A method of treating cancer in a patient in need thereof, comprising administering to the patient a therapeutically effective amount of the compound of any one of claims 1-24, or a pharmaceutically acceptable salt thereof, or a therapeutically effective amount of the pharmaceutical composition of claim 25.

27. The method of claim 26, wherein the cancer is liver, colorectal, anal, breast, gastrointestinal, skin, stomach, esophageal, or pancreatic cancer.

28. The method of claim 26 or 27, wherein the cancer originates from the liver or spreads to the liver.

29. The method of any one of claims 26-28, further comprising administering one or more additional pharmaceutical agents.

30. The method of claim 29, wherein the one or more additional pharmaceutical agents is selected from the group consisting of cabozantinib-S-malate, pembrolizumab, lenvatinib mesylate, sorafenib tosylate, nivolumab, and regorafenib.

31. The method of claim 29 or 30, wherein the one or more additional pharmaceutical agents is leucovorin.
